SEQUENCE LISTING (8 sequences)

SEQ ID NO: 1 - DNA, 1277 bp, Homo sapiens
aagagttgtctgagccgccgagtggacagtggctgattatggagagcagaggcccactggctacctcgcgcctgctgctgttgctgctgttgctactactgcgtcacacccgccagggatgggccctgagacctgttctccccacccagagtgcccacgaccctccggctgtccacctcagcaatggcccaggacaagagcctatcgctgtcatgacctttgacctcaccaagatcacaaaaacctcctcctcctttgaggttcgaacctgggacccagagggagtgattttttatggggataccaaccctaaggatgactggtttatgctgggacttcgagacggcaggcctgagatccaactgcacaatcactgggcccagcttacggtgggtgctggaccacggctggatgatgggagatggcaccaggtggaagtcaagatggagggggactctgtgctgctggaggtggatggggaggaggtgctgcgcctgagacaggtctctgggcccctgaccagcaaacgccatcccatcatgaggattgcgcttggggggctgctcttccccgcttccaaccttcggttgccgctggttcctgccctggatggctgcctgcgccgggattcctggctggacaaacaggccgagatctcagcatctgcccccactagcctcagaagctgtgatgtagaatcaaatcccgggatatttctccctccagggactcaggcagaattcaatctccgagacattccccagcctcatgcagagccctgggccttctctttggacctgggactcaagcaggcagcaggctcaggccacctccttgctcttgggacaccagagaacccatcttggctcagtctccacctccaagatcaaaaggtggtgttgtcttctgggtcggggccagggctggatctgcccctggtcttgggactccctcttcagctgaagctgagtatgtccagggtggtcttgagccaagggtcgaagatgaaggcccttgccctgcctcccttaggcctggctcccctccttaacctctgggccaagcctcaagggcgtctcttcctgggggctttaccaggagaagactcttccacctctttttgcctgaatggcctttgggcacaaggtcagaggctggatgtggaccaggccctgaacagaagccatgagatctggactcacagctgcccccagagcccaggcaatggcactgacgcttcccattaaagctccacctaagaaccccctttgaaagtt

SEQ ID NO: 2 - protein, 402 aa, Homo sapiens
MESRGPLATSRLLLLLLLLLLRHTRQGWALRPVLPTQSAHDPPAVHLSNGPGQEPIAVMTFDLTKITKTSSSFEVRTWDPEGVIFYGDTNPKDDWFMLGLRDGRPEIQLHNHWAQLTVGAGPRLDDGRWHQVEVKMEGDSVLLEVDGEEVLRLRQVSGPLTSKRHPIMRIALGGLLFPASNLRLPLVPALDGCLRRDSWLDKQAEISASAPTSLRSCDVESNPGIFLPPGTQAEFNLRDIPQPHAEPWAFSLDLGLKQAAGSGHLLALGTPENPSWLSLHLQDQKVVLSSGSGPGLDLPLVLGLPLQLKLSMSRVVLSQGSKMKALALPPLGLAPLLNLWAKPQGRLFLGALPGEDSSTSFCLNGLWAQGQRLDVDQALNRSHEIWTHSCPQSPGNGTDASH

SEQ ID NO: 3 - DNA, 1223 bp, Homo sapiens
aagagttgtctgagccgccgagtggacagtggctgattatggagagcagaggcccactggctacctcgcgcctgctgctgttgctgctgttgctactactgcgtcacacccgccagggatgggccctgagacctgttctccccacccagagtgcccacgaccctccggctgtccacctcagcaatggcccaggacaagagcctatcgctgtcatgacctttgacctcaccaagatcacaaaaacctcctcctcctttgaggttcgaacctgggacccagagggagtgattttttatggggataccaaccctaaggatgactggtttatgctgggacttcgagacggcaggcctgagatccaactgcacaatcactgggcccagcttacggtgggtgctggaccacggctggatgatgggagatggcaccaggtggaagtcaagatggagggggactctgtgctgctggaggtggatggggaggaggtgctgcgcctgagacaggtctctgggcccctgaccagcaaacgccatcccatcatgaggattgcgcttggggggctgctcttccccgcttccaaccttcggttgccggccgagatctcagcatctgcccccactagcctcagaagctgtgatgtagaatcaaatcccgggatatttctccctccagggactcaggcagaattcaatctccgagacattccccagcctcatgcagagccctgggccttctctttggacctgggactcaagcaggcagcaggctcaggccacctccttgctcttgggacaccagagaacccatcttggctcagtctccacctccaagatcaaaaggtggtgttgtcttctgggtcggggccagggctggatctgcccctggtcttgggactccctcttcagctgaagctgagtatgtccagggtggtcttgagccaagggtcgaagatgaaggcccttgccctgcctcccttaggcctggctcccctccttaacctctgggccaagcctcaagggcgtctcttcctgggggctttaccaggagaagactcttccacctctttttgcctgaatggcctttgggcacaaggtcagaggctggatgtggaccaggccctgaacagaagccatgagatctggactcacagctgcccccagagcccaggcaatggcactgacgcttcccattaaagctccacctaagaaccccctttgaaagtt

SEQ ID NO: 4 - protein, 384 aa, Homo sapiens
MESRGPLATSRLLLLLLLLLLRHTRQGWALRPVLPTQSAHDPPAVHLSNGPGQEPIAVMTFDLTKITKTSSSFEVRTWDPEGVIFYGDTNPKDDWFMLGLRDGRPEIQLHNHWAQLTVGAGPRLDDGRWHQVEVKMEGDSVLLEVDGEEVLRLRQVSGPLTSKRHPIMRIALGGLLFPASNLRLPAEISASAPTSLRSCDVESNPGIFLPPGTQAEFNLRDIPQPHAEPWAFSLDLGLKQAAGSGHLLALGTPENPSWLSLHLQDQKVVLSSGSGPGLDLPLVLGLPLQLKLSMSRVVLSQGSKMKALALPPLGLAPLLNLWAKPQGRLFLGALPGEDSSTSFCLNGLWAQGQRLDVDQALNRSHEIWTHSCPQSPGNGTDASH

SEQ ID NO: 5 - DNA, 1069 bp, Homo sapiens
aagagttgtctgagccgccgagtggacagtggctgattatggagagcagaggcccactggctacctcgcgcctgctgctgttgctgctgttgctactactgcgtcacacccgccagggatgggccctgagacctgttctccccacccagagtgcccacgaccctccggctgtccacctcagcaatggcccaggacaagagcctatcgctgtcatgacctttgacctcaccaagatcacaaaaacctcctcctcctttgaggttcgaacctgggacccagagggagtgattttttatggggataccaaccctaaggatgactggtttatgctgggacttcgagacggcaggcctgagatccaactgcacaatcactgggcccagcttacggtgggtgctggaccacggctggatgatgggagatggcaccaggtggaagtcaagatggagggggactctgtgctgctggaggtggatggggaggaggtgctgcgcctgagacaggtctctgggcccctgaccagcaaacgccatcccatcatgaggattgcgcttggggggctgctcttccccgcttccaaccttcggttgccgctggttcctgccctggatggctgcctgcgccgggattcctggctggacaaacaggccgagatctcagcatctgcccccactagcctcagaagctgtgatgtagaatcaaatcccgggatatttctccctccagggactcaggcagaattcaatctccgagacattccccagcctcatgcagagccctgggccttctctttggacctgggactcaagcaggcagcaggctcaggccacctccttgctcttgggacaccagagaacccatcttggctcagtctccacctccaagatcaagagaagactcttccacctctttttgcctgaatggcctttgggcacaaggtcagaggctggatgtggaccaggccctgaacagaagccatgagatctggactcacagctgcccccagagcccaggcaatggcactgacgcttcccattaaagctccacctaagaaccccctttgaaagtt

SEQ ID NO: 6 - protein, 293 aa, Homo sapiens
MESRGPLATSRLLLLLLLLLLRHTRQGWALRPVLPTQSAHDPPAVHLSNGPGQEPIAVMTFDLTKITKTSSSFEVRTWDPEGVIFYGDTNPKDDWFMLGLRDGRPEIQLHNHWAQLTVGAGPRLDDGRWHQVEVKMEGDSVLLEVDGEEVLRLRQVSGPLTSKRHPIMRIALGGLLFPASNLRLPLVPALDGCLRRDSWLDKQAEISASAPTSLRSCDVESNPGIFLPPGTQAEFNLRDIPQPHAEPWAFSLDLGLKQAAGSGHLLALGTPENPSWLSLHLQDQEKTLPPLFA

SEQ ID NO: 7 - DNA, 932 bp, Homo sapiens
aagagttgtctgagccgccgagtggacagtggctgattatggagagcagaggcccactggctacctcgcgcctgctgctgttgctgctgttgctactactgcgtcacacccgccagggatgggccctgagacctgttctccccacccagagtgcccacgaccctccggctgtccacctcagcaatggcccaggacaagagcctatcgctgtcatgacctttgacctcaccaagatcacaaaaacctcctcctcctttgaggttcgaacctgggacccagagggagtgattttttatggggataccaaccctaaggatgactggtttatgctgggacttcgagacggcaggcctgagatccaactgcacaatcactgggcccagcttacggtgggtgctggaccacggctggatgatgggagatggcaccaggtggaagtcaagatggagggggactctgtgctgctggaggtggatggggaggaggtgctgcgcctgagacaggtctctgggcccctgaccagcaaacgccatcccatcatgaggattgcgcttggggggctgctcttccccgcttccaaccttcggttgccgctggttcctgccctggatggctgcctgcgccgggattcctggctggacaaacaggccgagatctcagcatctgcccccactagcctcagaagctgtgatgtagaatcaaatcccgggatatttctccctccagggactcaggcagaattcaatctccgaggagaagactcttccacctctttttgcctgaatggcctttgggcacaaggtcagaggctggatgtggaccaggccctgaacagaagccatgagatctggactcacagctgcccccagagcccaggcaatggcactgacgcttcccattaaagctccacctaagaaccccctttgaaagtt

SEQ ID NO: 8 - protein, 287 aa, Homo sapiens
MESRGPLATSRLLLLLLLLLLRHTRQGWALRPVLPTQSAHDPPAVHLSNGPGQEPIAVMTFDLTKITKTSSSFEVRTWDPEGVIFYGDTNPKDDWFMLGLRDGRPEIQLHNHWAQLTVGAGPRLDDGRWHQVEVKMEGDSVLLEVDGEEVLRLRQVSGPLTSKRHPIMRIALGGLLFPASNLRLPLVPALDGCLRRDSWLDKQAEISASAPTSLRSCDVESNPGIFLPPGTQAEFNLRGEDSSTSFCLNGLWAQGQRLDVDQALNRSHEIWTHSCPQSPGNGTDASH

(19) United States
(12) Patent Application Publication
(10) Pub. No.: US 2012/0157378 A1
(43) Pub. Date: Jun. 21, 2012

(54) Methods and Compositions for Predicting a Subject's Susceptibility To and Risk of Developing Type 2 Diabetes

(76) Inventors: Simin Liu, Calabasas, CA (US); Eric L. Ding, Jamaica Plain, MA (US); Joann E. Manson, Beverly, MA (US)

(21) Appl. No.: 13/125,961

(22) PCT Filed: Nov. 16, 2009
(86) PCT No.: PCT/US09/64656

§ 371 Date: Mar. 2, 2012

(30) Foreign Application Priority Data
Nov. 14, 2008 (US) 61/114963

(51) Int. Cl. C12Q 1/68; A61P 3/10; A61K 31/7088; A61K 38/02; G01N 33/566; C12M 1/40

(52) U.S. Cl. 514/6.9; 436/501; 435/6.11; 435/287.2; 514/44 R; 422/69

(57) ABSTRACT

Aspects of the invention include methods of predicting a subject's susceptibility to developing type 2 diabetes. Embodiments of the methods include obtaining a sex hormone-binding globulin (SHBG) level value for the subject, e.g., by detecting a SHBG plasma concentration and/or a SHBG polymorphism phenotype, and predicting the subject's susceptibility to developing type 2 diabetes from the obtained SHBG level value. Also provided are devices and kits that find use in practicing embodiments of the methods. In addition, methods of treating a subject for type 2 diabetes and/or preventing the onset of type 2 diabetes are provided.

### CROSS-REFERENCE TO RELATED APPLICATIONS

Pursuant to 35 U.S.C. §119 (e), this application claims priority to the filing date of the U.S. Provisional Patent Application Ser. No. 61/114,963 filed Nov. 14, 2008; the disclosure of which is herein incorporated by reference.

This invention was made with Government support under Grant Nos. R01-DK066401 awarded by the National Institute of Health. The Government has certain rights in this invention.

### INTRODUCTION

Diabetes mellitus type 2, or type 2 diabetes (formerly called non-insulin-dependent diabetes mellitus (NIDDM), or adult-onset diabetes), is a disorder that is characterized by high blood glucose in the context of insulin resistance and relative insulin deficiency. There are an estimated 23.6 million people in the U.S. (7.8% of the population) with diabetes with 17.9 million being diagnosed, 90% of whom are type 2. With prevalence rates doubling between 1990 and 2005, CDC has characterized the increase as an epidemic. The elucidation of biomarkers that may be used to predict susceptibility to developing type 2 diabetes and of novel therapeutics to treat type 2 diabetes are therefore of great clinical interest. The present invention addresses these issues.

### SUMMARY

Aspects of the invention include methods of predicting a subject's susceptibility to developing type 2 diabetes and of predicting a subject's risk of developing type 2 diabetes. Embodiments of the methods include obtaining a sex hormone-binding globulin (SHBG) plasma level value, that is an SHBG level value, for the subject, e.g., by detecting a SHBG concentration and/or a SHBG polymorphism genotype, and predicting the subject's susceptibility to developing type 2 diabetes from the obtained SHBG level value. Also provided are devices and kits that find use in practicing embodiments of the methods. In addition, methods of treating a subject for type 2 diabetes and/or preventing the onset of type 2 diabetes are provided.

### BRIEF DESCRIPTION OF THE FIGURES

FIG. 1 provides the coding sequence of the Sex Hormone-Binding Globulin (SHBG) variant/isoform 1 (SEQ ID NO:1) and encoded polypeptide (SEQ ID NO:2).

FIG. 2 provides the coding sequence of the Sex Hormone-Binding Globulin (SHBG) variant/isoform 1 (SEQ ID NO:3) and encoded polypeptide (SEQ ID NO:4).

FIG. 3 provides the coding sequence of the Sex Hormone-Binding Globulin (SHBG) variant/isoform 1 (SEQ ID NO:5) and encoded polypeptide (SEQ ID NO:6).

FIG. 4 provides the coding sequence of the Sex Hormone-Binding Globulin (SHBG) variant/isoform 1 (SEQ ID NO:7) and encoded polypeptide (SEQ ID NO:8).

FIG. 5 provides results of levels of Sex Hormone-Binding Globulin (SHBG) and risk of type 2 diabetes in women, according to SHBG genotypes. Panel A depicts percent changes in SHBG levels for each of three variant-genotype groups as compared with carriers of the rs6257 variant allele, who were also homozygous for the rs6259 wild-type allele (associated with the lowest SHBG level). Panel B depicts odds of type 2 diabetes among the same genotype groups. The 95% confidence intervals are given in parentheses.

FIG. 6 provides C-statistics and ROC curves comparing SHBG vs. traditional risk factors, C-reactive protein (CRP), and HbA1c in women. Panel A depicts traditional risk factors plus SHBG versus traditional risk factors alone. Panel B depicts traditional risk factors plus CRP plus SHBG versus traditional risk factors plus CRP. Panel C depicts traditional risk factors plus HbA1c (glycated hemoglobin) plus SHBG versus Traditional Risk Factors plus HbA1c. Panel D depicts traditional risk factors plus CRP plus HbA1c plus SHBG versus traditional risk factors plus CRP plus HbA1c.

### DETAILED DESCRIPTION

Aspects of the invention include methods of predicting a subject's susceptibility to developing type 2 diabetes. Embodiments of the methods include obtaining a sex hormone-binding globulin (SHBG) level value, that is, an SHBG level, for the subject, e.g., by detecting a SHBG plasma concentration and/or a SHBG polymorphism phenotype, and predicting the subject's susceptibility to developing type 2 diabetes from the obtained SHBG level value. Also provided are devices and kits that find use in practicing embodiments of the methods. In addition, methods of treating a subject for type 2 diabetes and/or preventing the onset of type 2 diabetes are provided.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As summarized above, the subject invention is directed to methods of predicting whether a subject is susceptible to or at risk of developing type 2 diabetes, methods of treating a subject for type 2 diabetes and method of preventing the onset of type 2 diabetes, as well as reagents and kits thereof (and devices) for use in practicing the subject methods. In further describing the invention, the subject methods are described first, followed by a review of the reagents, kits and devices for use in practicing the subject methods.

### Methods of Predicting a Subject's Susceptibility to Developing Type 2 Diabetes

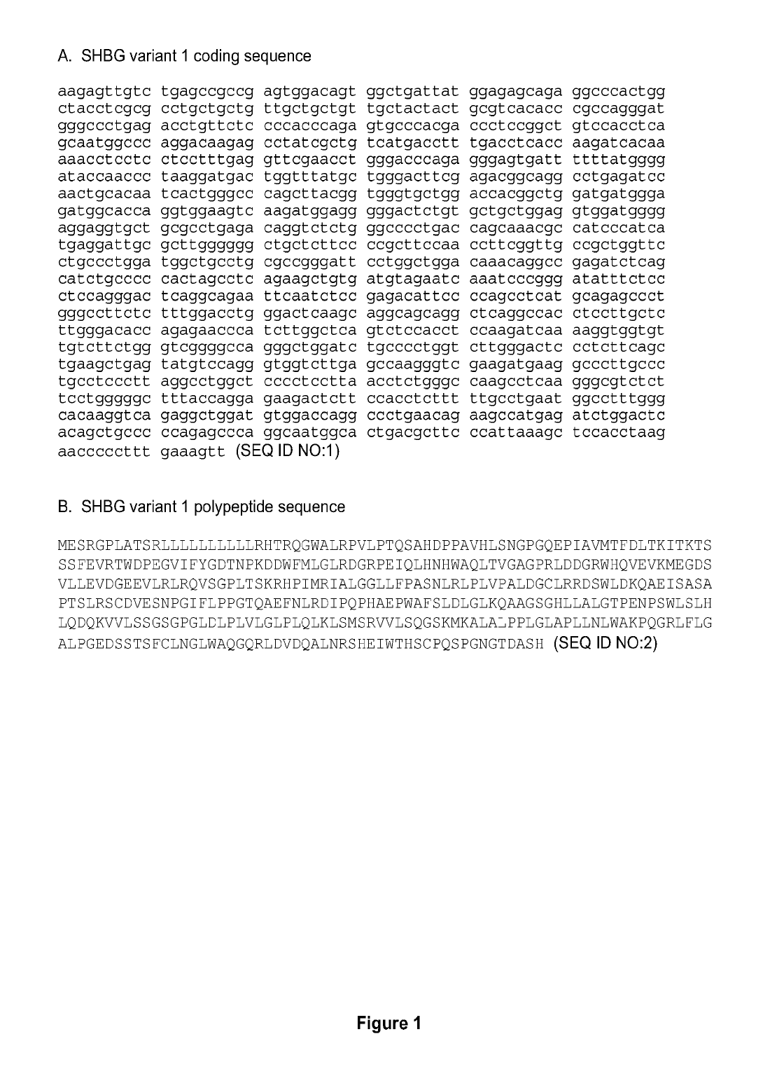

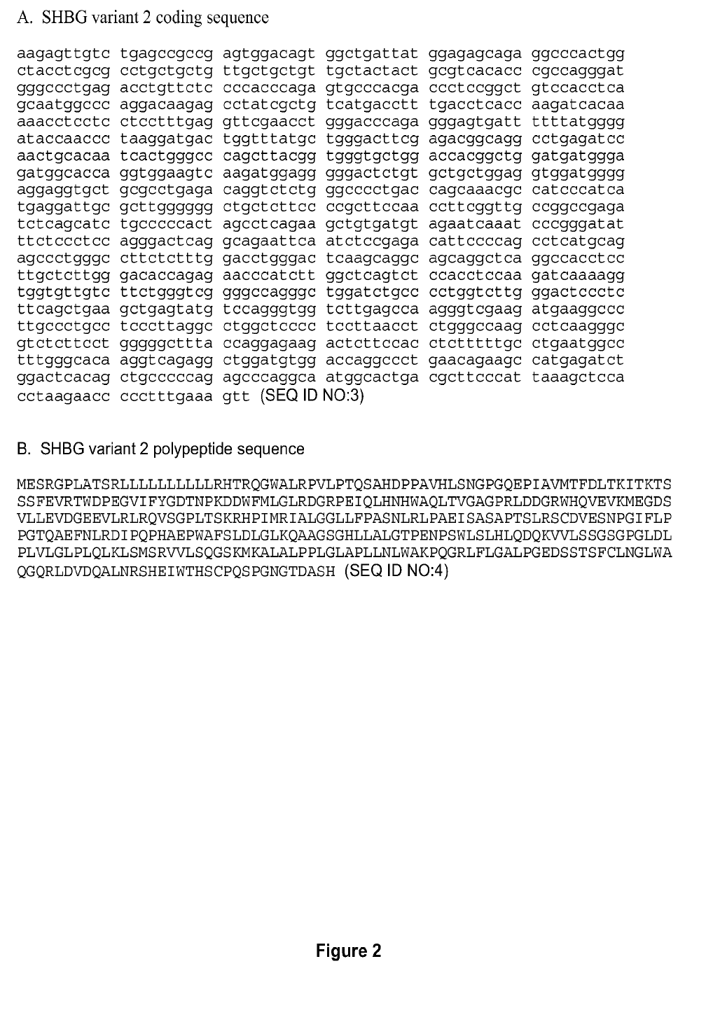

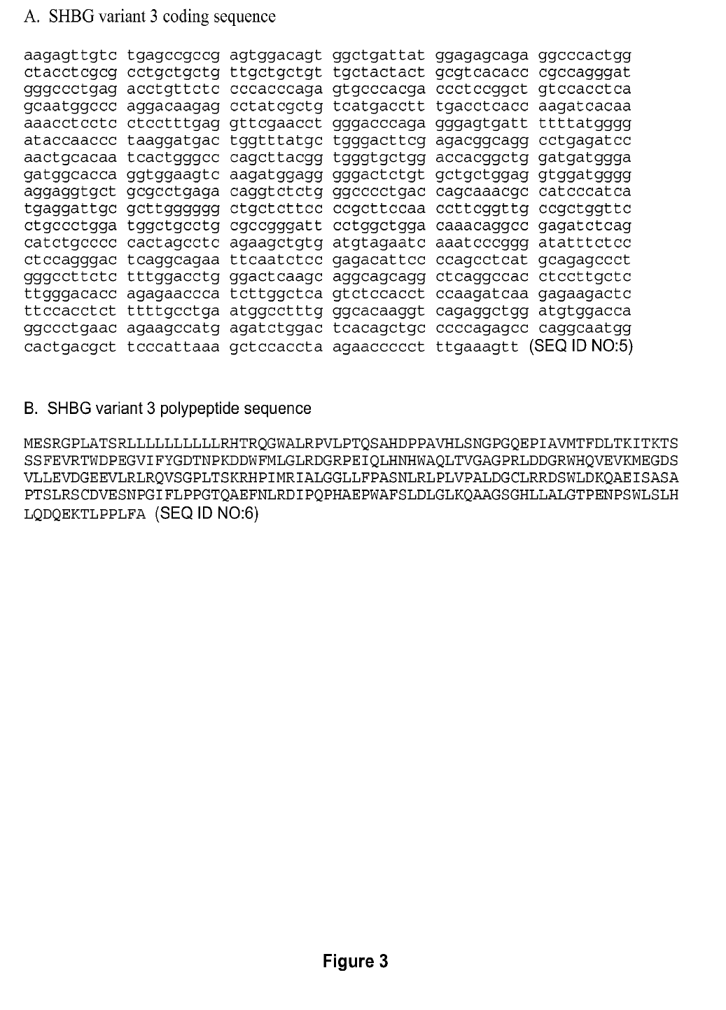

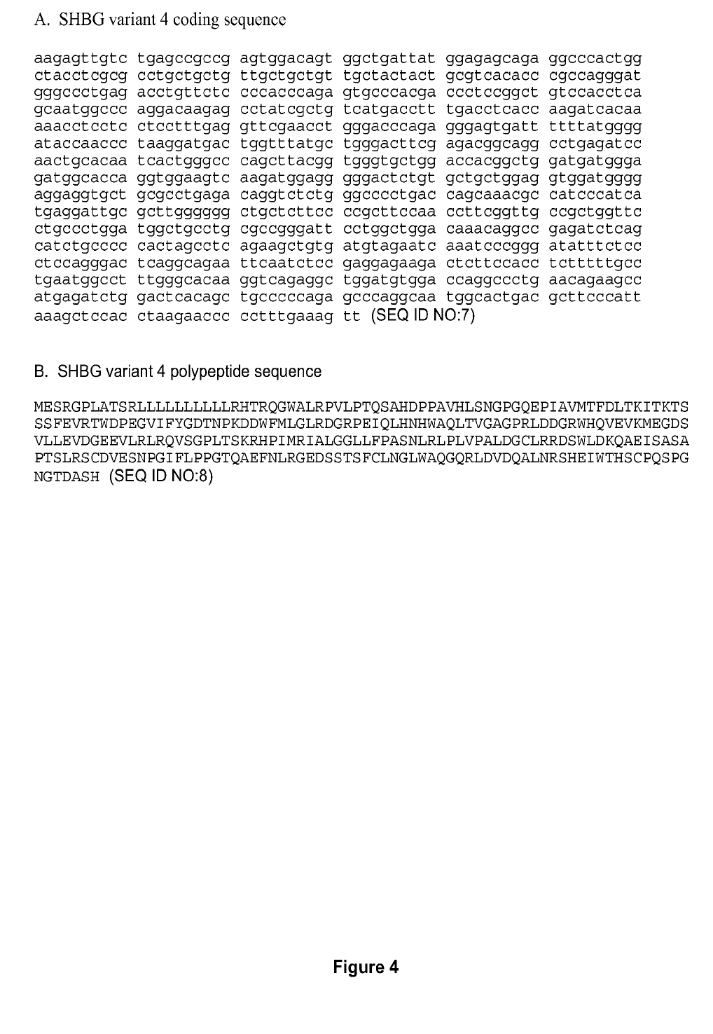

The subject invention provides methods of predicting whether a subject will develop type 2 diabetes. In practicing the subject methods, a sex hormone-binding globulin (SHBG) level value for the subject is obtained. A SHBG level value is a value or number that signifies, i.e. represents, the plasma concentration of SHBG for the subject. SHBG is a glycoprotein that binds circulating sex steroid hormones such as testosterone and estradiol. Four variants, or isoforms, exist. The genetic sequences encoding these 4 isoforms may be found in FIGS. 1-4; more information regarding these sequence may be found at Genbank accession numbers NM_{—}001040, NM_{—}001146279, NM_{—}001146280, and NM_{—}001146281. In some embodiments, the SHBG level value is a SHBG plasma concentration. In some embodiments, the SHBG level value is a SHBG polymorphism genotype, where the polymorphism genotype correlates with a SHBG plasma concentration or SHBG activity.

In some embodiments, the SHBG level value is obtained by obtaining a fluid sample, such as blood or a fraction/derivative thereof, e.g., plasma or serum, from the subject and determining, i.e., measuring, the SHBG concentration, that is, the concentration of SHBG polypeptide, in the fluid sample. Any convenient method for producing a fluid sample may be employed. In many embodiments, the fluid sample is a blood sample, produced for example by drawing venous blood by skin puncture (e.g., finger stick, venipuncture). In some embodiments, the cells in the blood sample are lysed or otherwise removed from the sample so as to prepare a plasma sample. In some embodiments, clotting factors, e.g., fibrinogen, clotting proteins, etc., are removed from the sample e.g., by allowing the blood to clot, e.g., in a clotting or serum separator tube, and centrifuging the serum away from the clotted blood, so as to prepared a serum sample.

The fluid sample may be assayed for SHBG concentration at the time of collection/preparation or stored, e.g. at 4° C., at −20° C., at −60° C., at −80° C. for assaying at a later time. The fluid may be assayed in crude form. Alternatively, the fluid may be fractionated to purify the SHBG polypeptide based upon size, charge, mass, or other physical characteristic prior to measuring SHBG concentration. If fractionation is employed, the fractionation technique may or may not employ native, i.e., non-denaturing, conditions. Whether fractionation is carried out under denaturing or non-denaturing conditions depends on the particular manner in which the SHBG plasma concentration is detected, e.g., whether or not a non-denatured form of protein is required for detection, where representative detection methods are described in greater detail below. Typically, the non-denaturing, or native, conditions substantially preserve the conformation and folding of the subject polypeptide in the sample. A variety of non-denaturing fractionation protocols are known to those of skill in the art, e.g., gel filtration high performance liquid chromatography (HPLC). Alternatively, fractionation may be carried out under non-native, that is, denaturing conditions, such as sodium dodecylsulfate-polyacrylamide gel electrophoresis (SDS-PAGE). Where desired, one or more fractions are then assayed to quantitate the amount of protein. Alternatively, the fluid is not fractionated, but rather is assayed in crude form.

Any convenient assay protocol for assaying protein concentration in a fluid sample may be employed. Suitable assays include specific binding member assays, i.e., ligand-based assays, in which a ligand that specifically binds to a SHBG is employed. Ligands of interest may vary, and include antibodies and binding fragments thereof, aptamers, etc. Ligands of interest may have an affinity for SHBG of 10⁻⁴ M or greater, such as 10⁻⁶ molar or greater and 10⁻⁸ M or greater, where in some embodiments the ligand has an affinity for SHBG of between 10⁻⁹ and 10⁻¹² M.

In some instances, the assay is an immunoassay (i.e., antibody-based assay), such that it employs an antibody or binding fragment thereof. Antibody-based assays use of antibodies (or binding fragments thereof) specific for a polypeptide of interest, i.e., such as SHBG polypeptide or fragments thereof. Antibodies that specifically bind to SHBG can be prepared using a variety of convenient methods known to those of skill in the art. See Guide to Protein Purification, supra, as well as Antibodies, A Laboratory Manual (Harlow & Lane eds. Cold Spring Harbor Press, 1988). The antibodies may be polyclonal or monoclonal antibodies depending on the nature of the intended use, as long as they are specific for one or more forms of SHBG or fragments thereof of interest. Antibodies specific for SHBG polypeptide are known in the art, and include sc32467, sc32467, and sc32890 (Santa Cruz Biotechnology, Inc); anti-SHBG 6462-A01 (Novus Biologicals); and AF2656 (R&S Systems), etc. In some instances, these known in the art antibodies are employed.

In specific binding member assays of the subject invention, a number of different formats are known in the art and may be employed. For example, a sandwich assay may be employed. A sandwich assay is performed by initially attaching a “capture agent”, that is, a ligand that specifically binds to SHBG, e.g., a hormone which binds SHBG, an SHBG-specific antibody, etc., to an insoluble surface or support. This capture agent may be bound to the surface by any convenient means, depending upon the nature of the surface, either directly or through specific antibodies; the particular manner of binding is not crucial so long as it is compatible with the reagents and overall methods of the invention. The capture agent may be bound to the plates covalently or non-covalently. The insoluble supports may be any compositions to which polypeptides can be bound, which is readily separated from soluble material, and which is otherwise compatible with the overall method of measuring subject polypeptide in the sample. The surface of such supports may be solid or porous and of any convenient shape. Examples of suitable insoluble supports to which the receptor is bound include beads, e.g. magnetic beads, membranes and microtiter plates. Materials of interest include glass, plastic (e.g. polystyrene), polysaccharides, nylon or nitrocellulose. Microtiter plates are especially convenient because a large number of assays can be carried out simultaneously, using small amounts of reagents and samples. Before adding patient samples or fractions thereof, the non-specific binding sites on the insoluble support i.e., those not occupied by the capture agent, are generally blocked. Blocking agents of interest include non-interfering proteins such as bovine serum albumin, casein, gelatin, and the like.

Patient sample preparations are then added to the capture agent-bound substrate. In some instances, a series of standards containing known concentrations of the test protein may be assayed in parallel with the samples or aliquots thereof to serve as controls. Samples may be assayed in multiple spots, wells, etc., so that mean values can be obtained for each. Incubation times may vary so long as they are sufficient for desired binding to occur, and in some instances may range from 0.1 to 3 hr. After incubation, the insoluble support may be washed of non-bound components, where desired. A dilute non-ionic detergent medium at an appropriate pH, such as 7-8, can, be used as a wash medium. When the support is washed, the number of washes may vary, where in certain instances from one to six washes can be employed, with sufficient volume to thoroughly wash non-specifically bound proteins present in the sample.

After washing, a solution containing a “detection agent”, e.g. antibodies reactive with the subject polypeptide, i.e. SHBG-specific antibodies, may be applied to the sample contacted support. The detection agent may be labeled to facilitate direct or indirect quantification of binding. Examples of labels that permit direct measurement of detection agent binding include radiolabels, such as ³H or ¹²⁵I, fluorescers, dyes, beads, chemiluminescers, colloidal particles, and the like. Examples of labels that permit indirect measurement of binding include enzymes where the substrate may provide for a colored or fluorescent product. In a preferred embodiment, the detection agent is labeled with a covalently bound enzyme capable of providing a detectable product signal after addition of suitable substrate. Examples of suitable enzymes for use in conjugates include horseradish peroxidase, alkaline phosphatase, malate dehydrogenase and the like. The incubation time may be chosen to be sufficient for the labeled detection agent to bind available molecules. In some instances, incubation times ranging from 0.1 to 3 hrs, such as 0.5 to 1 hr, are employed.

After the second binding step, the insoluble support may again be washed free of non-specifically bound material, leaving the specific complex formed between the subject's SHBG polypeptides and the detection agent. The signal produced by the bound conjugate may be detected using any convenient protocol, e.g., using a scanner, etc. Where an enzyme conjugate is used, an appropriate enzyme substrate is provided so a detectable product is formed.

Other types of ligand based assays may also find use in determining the concentration of SHBG polypeptide in a fluid sample. For example, Western blots may be performed on protein gels or protein spots on filters, using a detection system specific for the subject polypeptide as desired, conveniently using a labeling method as described for the sandwich assay above. See, for example, Lewis, J G et al (1999) Steroids 64(4):259-265, which describes Western blotting assays to measure SHBG concentrations in fluid samples, and is incorporated herein by reference.

Other ligand based assays of interest include those based on competitive formats. One such format would be where a solid support is coated with an amount of SHBG. Labeled ligand that specifically binds to SHBG, such as labeled SHBG antibodies or binding fragments thereof, is then combined with the patient derived sample which, following sufficient incubation time for binding complexes to form, is contacted with the solid phase bound subject polypeptide. The amount of labeled antibody which binds to the solid phase will be proportional to the amount of subject polypeptide or fragments thereof in the sample, and the presence of subject polypeptide and fragments thereof may therefore be detected. Other competitive formats that may be employed include those where the sample suspected of comprising subject polypeptide is combined with a known amount of labeled subject polypeptide or fragments thereof and then contacted with a solid support coated with antibody specific for the subject polypeptide. Such assay formats are further described in both Guide to Protein Purification, supra, and Antibodies, A Laboratory Manual, supra.

While the detected SHBG level may vary depending on the particular patient for which it is assessed, in some instances the level that is detected will range from less than 1 nmol/liter to 200 nmol/liter, such as 4 nmol/liter to 150 nmol/liter and including 4.4 nmol/liter to 122 nmol/liter.

In some embodiments, the SHBG level value is obtained by genotyping the subject for an SHBG polymorphism. SHBG polymorphisms of interest include the rs6257 and rs6259 polymorphisms. Where a subject is genotyped for an SHBG polymorphism, a subject or patient sample, e.g., cells or collections thereof, e.g., tissues, is assayed to determine the nucleotide sequence of the gene at that polymorphism, the amino acid sequence encoded by the gene at that polymorphism, or the concentration of the encoded SHBG protein, e.g., by using one or more genotyping reagents, such as but not limited to nucleic acid reagents, including primers, etc., which may or may not be labeled, as described below, amplification enzymes, buffers, etc. In practicing the subject prognostic methods, the sample obtained from the subject is assayed to determine the genotype of the subject from which the sample was obtained with respect to at least one, i.e., one or more, polymorphisms, where polymorphisms of interest are referred to herein as target polymorphisms, examples of which are mentioned above. Any convenient protocol for assaying a sample for the above one or more target polymorphisms may be employed in the subject methods. In certain embodiments, the target polymorphism will be detected at the protein level, e.g., by assaying for a polymorphic protein. In yet other embodiments, the target polymorphism will be detected at the nucleic acid level, e.g., by assaying for the presence of nucleic acid polymorphism, e.g., an single nucleotide polymorphism (SNP) that cause expression of the polymorphic protein.

For example, polynucleotide samples derived from (e.g., obtained from) an individual may be employed. Any biological sample that comprises a polynucleotide from the individual is suitable for use in the methods of the invention. The biological sample may be processed so as to isolate the polynucleotide. Alternatively, whole cells or other biological samples may be used without isolation of the polynucleotides contained therein. Detection of a target polymorphism in a polynucleotide sample derived from an individual can be accomplished by any means known in the art, including, but not limited to, amplification of a sequence with specific primers; determination of the nucleotide sequence of the polynucleotide sample; hybridization analysis; single strand conformational polymorphism analysis; denaturing gradient gel electrophoresis; mismatch cleavage detection; and the like. Detection of a target polymorphism can also be accomplished by detecting an alteration in the level of a mRNA transcript of the gene; aberrant modification of the corresponding gene, e.g., an aberrant methylation pattern; the presence of a non-wild-type splicing pattern of the corresponding mRNA; an alteration in the level of the corresponding polypeptide; and/or an alteration in corresponding polypeptide activity.

Detection of a target polymorphism by analyzing a polynucleotide sample can be conducted in a number of ways. A test nucleic acid sample can be amplified with primers which amplify a region known to comprise the target polymorphism(s). Genomic DNA or mRNA can be used directly. Alternatively, the region of interest can be cloned into a suitable vector and grown in sufficient quantity for analysis. The nucleic acid may be amplified by conventional techniques, such as a polymerase chain reaction (PCR), to provide sufficient amounts for analysis. The use of the polymerase chain reaction is described in a variety of publications, including, e.g., “PCR Protocols (Methods in Molecular Biology)” (2000) J. M. S. Bartlett and D. Stirling, eds, Humana Press; and “PCR Applications: Protocols for Functional Genomics” (1999) Innis, Gelfand, and Sninsky, eds., Academic Press. Once the region comprising a target polymorphism has been amplified, the target polymorphism can be detected in the PCR product by nucleotide sequencing, by SSCP analysis, or any other method known in the art. In performing SSCP analysis, the PCR product may be digested with a restriction endonuclease that recognizes a sequence within the PCR product generated by using as a template a reference sequence, but does not recognize a corresponding PCR product generated by using as a template a variant sequence by virtue of the fact that the variant sequence no longer contains a recognition site for the restriction endonuclease.

PCR may also be used to determine whether a polymorphism is present by using a primer that is specific for the polymorphism. Such methods may comprise the steps of collecting from an individual a biological sample comprising the individual's genetic material as template, optionally isolating template nucleic acid (genomic DNA, mRNA, or both) from the biological sample, contacting the template nucleic acid sample with one or more primers that specifically hybridize with a target polymorphic nucleic acid molecule under conditions such that hybridization and amplification of the template nucleic acid molecules in the sample occurs, and detecting the presence, absence, and/or relative amount of an amplification product and comparing the length to a control sample. Observation of an amplification product of the expected size is an indication that the target polymorphism contained within the target polymorphic primer is present in the test nucleic acid sample. Parameters such as hybridization conditions, polymorphic primer length, and position of the polymorphism within the polymorphic primer may be chosen such that hybridization will not occur unless a polymorphism present in the primer(s) is also present in the sample nucleic acid. Those of ordinary skill in the art are well aware of how to select and vary such parameters. See, e.g., Saiki et al. (1986) Nature 324:163; and Saiki et al (1989) Proc. Natl. Acad. Sci. USA 86:6230.

Alternatively, various methods are known in the art that utilize oligonucleotide ligation as a means of detecting polymorphisms. See, e.g., Riley et al. (1990) Nucleic Acids Res. 18:2887-2890; and Delahunty et al. (1996) Am. J. Hum. Genet. 58:1239-1246.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid may be sequenced using any convenient sequencing protocol, such as a dideoxy chain termination method protocol. Genomic DNA or mRNA may be used directly. If mRNA is used, a cDNA copy may first be made. If desired, the sample nucleic acid can be amplified using a PCR. A variety of sequencing reactions known in the art can be used to directly sequence the relevant gene, or a portion thereof in which a specific polymorphism is known to occur, and detect polymorphisms by comparing the sequence of the sample nucleic acid with a reference polynucleotide that contains a target polymorphism. Any of a variety of automated sequencing procedures can be used. See, e.g., WO 94/16101; Cohen et al. (1996) Adv. Chromatography 36:127-162.

Hybridization with the variant sequence may also be used to determine the presence of a target polymorphism. Hybridization analysis can be carried out in a number of different ways, including, but not limited to Southern blots, Northern blots, dot blots, microarrays, etc. The hybridization pattern of a control and variant sequence to an array of oligonucleotide probes immobilized on a solid support, as described in U.S. Pat. No. 5,445,934, or in WO 95/35505, may also be used protocols for detecting the presence of variant sequences. Identification of a polymorphism in a nucleic acid sample can be performed by hybridizing a sample and control nucleic acids to high density arrays containing hundreds or thousands of oligonucleotide probes. Cronin et al. (1996) Human Mutation 7:244-255; and Kozal et al. (1996) Nature Med. 2:753-759.

Single strand conformational polymorphism (SSCP) analysis; denaturing gradient gel electrophoresis (DGGE); mismatch cleavage detection; and heteroduplex analysis in gel matrices can also be used to detect polymorphisms. Alternatively, where a polymorphism creates or destroys a recognition site for a restriction endonuclease (restriction fragment length polymorphism, RFLP), the sample is digested with that endonuclease, and the products size fractionated to determine whether the fragment was digested. Fractionation is performed by gel or capillary electrophoresis, particularly acrylamide or agarose gels. The aforementioned techniques are well known in the art. Detailed description of these techniques can be found in a variety of publications, including, e.g., “Laboratory Methods for the Detection of Mutations and Polymorphisms in DNA” (1997) G. R. Taylor, ed., CRC Press, and references cited therein.

A number of methods are available for determining the expression level of a polymorphic nucleic acid molecule, e.g., a polymorphic mRNA or polymorphic polypeptide in a particular sample. Diagnosis may be performed by a number of methods to determine the amounts of normal or abnormal mRNA in a patient sample. For example, detection may utilize staining of cells or histological sections with labeled antibodies, performed in accordance with conventional methods. Cells are permeabilized to stain cytoplasmic molecules. The antibodies of interest are added to the cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Alternatively, the secondary antibody conjugated to a fluorescent compound, e.g. fluorescein, rhodamine, Texas red, etc. Final detection uses a substrate that undergoes a color change in the presence of the peroxidase. The absence or presence of antibody binding may be determined by various methods, including flow cytometry of dissociated cells, microscopy, radiography, scintillation counting, etc.

Alternatively, one may focus on the expression of mRNA. Biochemical studies may be performed to determine whether a sequence polymorphism in a SHBG coding region or control regions is associated with disease. Disease associated polymorphisms may include deletion or truncation of the gene, mutations that alter expression level, that affect the activity of the protein, etc.

Screening for mutations in a polymorphic polypeptide may be based on the functional or antigenic characteristics of the protein. Protein truncation assays are useful in detecting deletions that may affect the biological activity of the protein. Various immunoassays designed to detect polymorphisms in polymorphic polypeptides may be used in screening. Where many diverse genetic mutations lead to a particular disease phenotype, functional protein assays have proven to be effective screening tools. The activity of the encoded a polymorphic polypeptide may be determined by comparison with a reference polypeptide lacking a specific polymorphism.

Prognostic methods of the subject invention in which the level of polymorphic gene expression is of interest may involve comparison of the relevant nucleic acid abundance of a sample of interest with that of a control value to determine any relative differences, where the difference may be measured qualitatively and/or quantitatively, which differences are then related to the presence or absence of an abnormal gene expression pattern. Methods of interest for determining the nucleic acid abundance in a sample include those described in: Pietu et al., Genome Res. (June 1996) δ: 492-503; Zhao et al., Gene (Apr. 24, 1995) 156: 207-213; Soares, Curr. Opin. Biotechnol. (October 1997) 8: 542-546; Raval, J. Pharmacol Toxicol Methods (November 1994) 32: 125-127; Chalifour et al., Anal. Biochem (Feb. 1, 1994) 216: 299-304; Stolz & Tuan, Mol. Biotechnol. (December 19960 6: 225-230; Hong et al., Bioscience Reports (1982) 2: 907; and McGraw, Anal. Biochem. (1984) 143: 298. Also of interest are the methods disclosed in WO 97/27317, the disclosure of which is herein incorporated by reference.

Additional references describing various protocols for detecting the presence of a target polymorphism include, but are not limited to, those described in: 6,703,228; 6,692,909; 6,670,464; 6,660,476; 6,653,079; 6,632,606; 6,573,049; the disclosures of which are herein incorporated by reference.

As discussed above, SHBG polymorphisms of interest include the rs6257 and rs6259 polymorphisms. In some instances, what is detected is the wild type allele for the polymorphism, that is, T for rs6257, e.g., in an individual that is TT or CT for a rs6257 polymorphism; or G for rs6259, e.g., in an individual that is GG or AG for a rs6259 polymorphism. In some instances, what is detected is the variant allele for the polymorphism, that is, C for rs6257, e.g., in a subject that is CT or CC for the rs6257 polymorphism; or A for rs6259, e.g., in a subject that is AG or AA for the rs6259 polymorphism. In some cases, both alleles may be detected, that is, TT, CT or CC for rs6257, or GG, AG or AA for rs6259.

In certain embodiments, the methods include both determining a sample SHBG concentration and genotyping the subject for a SHBG polymorphism. Such methods find use in, for example, fine-tuning the risk prediction for a subject's susceptibility to developing type 2 diabetes and for increasing the confidence indicia of those risk predictions. Such methods can be performed on the same sample, for example by separating DNA from protein, e.g., by precipitation or phenol-based extraction (e.g., Trizol™), then assaying each fraction by the above methods. Alternatively, the sample as a whole may be divided into two fractions such that each fraction comprises all the components of the sample, where one fraction is assayed for SHBG concentration and the other fraction is assayed for SHBG polymorphism genotype. One example of a device for use in these embodiments is a multifunctional biosensor device, e.g., a multiplex assay platform, which contains a multifunctional array programmably coupled to an array of biosensor devices, each configured to obtain a SHBG level value by a different means, e.g., a protein or peptide sensor for determining SHBG concentration; a DNA, RNA or protein sensor for genotyping an SHBG polymorphism; etc. Such platforms may optionally be configured to include additional biosensor devices, e.g. a steroid sensor, a lipid sensor, etc. The biosensors obtain the SHBG level value by methods such as those described above. In some embodiments, the biosensor then outputs the data to an integrated network that applies systems-biology software to assess the SHGB level value, that is, the SHBG concentration or SHBG polymorphism nucleotide or polypeptide sequence, and provide a prediction as to the subject's susceptibility to developing type 2 diabetes. Examples of multiplex assay platforms and methods for there use that may be tailored for use in methods of the present invention by incorporating components that provide for specific detection of SHBG and/or SHBG polymorphisms (such as described above) are provided in US Application No. 20050043894, US Application No. 20060253259, and US Application No. 20070106333; the disclosures of which are incorporated herein by reference.

The obtained SHBG level value for a subject is employed to make a prediction (e.g., a prognosis) regarding the subject's susceptibility to, that is, propensity to, likelihood of, or risk of developing, type 2 diabetes. By prediction it is meant assessing an obtained value and providing a hypothesis regarding an outcome, i.e., assessing an obtained SHBG plasma value and providing a hypothesis regarding a subject's susceptibility to developing type 2 diabetes. In some embodiments, the obtained SHBG level value is assessed by comparing the obtained SHBG level value to one or more reference or control values. The terms “reference” and “control” as used herein refer to a predetermined SHBG level value to be used to interpret the SHBG level value of a given subject and assign a prediction of susceptibility for developing type 2 diabetes thereto. For example, when the SHBG level value is obtained by genotyping an SHBG polymorphism, the reference value may be the polymorphism genotype of a particular subpopulation of the general population. Or, when the SHBG level value is obtained by determining the SHBG concentration in the fluid, the reference value may be the SHBG concentration obtained in a particular subpopulation of individuals e.g., a population of individuals with a particularly low concentration or a particularly high concentration of SHBG. A table of values may be consulted in this step, where the table comprises one or more reference values for which risks of developing type 2 diabetes have been predetermined, e.g., particular SHBG polymorphism genotypes, or median SHBG concentrations or ranges of SHBG concentrations. The values may be presented in numerical form, in picture form (e.g., as bands on a gel), and the like. By comparing the observed value with these reference values, e.g., by comparing the SHBG level value in the sample to a reference pattern or picture, the obtained SHBG level value may be assessed such that predictions of susceptibility to disease or efficacy of treatment may be readily made.

In some instances, the method is used to predict a reduced susceptibility to developing type 2 diabetes. By a reduced susceptibility to developing type 2 diabetes, it is meant that the subject may be predicted to have about 66%, about 50%, about 40%, about 30%, about 25%, about 20%, about 15%, about 10%, or about 5% the risk of developing type 2 diabetes as other individuals of the general population. In other words, the subject may be predicted to be 1.5-fold (i.e. 1.5 times) less likely, 2-fold less likely, 2.5-fold less likely, 3-fold less likely, 4-fold less likely, 5-fold less likely, 7-fold less likely, 10-fold less likely, or 20-fold less likely to develop type 2 diabetes than other individuals of the general population. In other instances, the method is used to predict an increased susceptibility to developing type 2 diabetes. By increased susceptibility to developing type 2 diabetes, it is meant that the subject may be predicted to have 150% or more, 200% or more, 250% or more, 300% or more, 500% or more, or 1000% or more the risk of developing type 2 diabetes as other individuals of the general population. In other words, the subject may be predicted to be 1.5-fold (i.e., 1.5 times), 2-fold, 2.5-fold, 3-fold, 5-fold, or 10-fold or more likely to develop type 2 diabetes than other individuals of the general population.

For example, in some embodiments, the method includes predicting the susceptibility to developing diabetes based upon an SHBG polymorphism. In certain embodiments, an increased susceptibility to developing type 2 diabetes is predicted if the subject carries at least one C allele of the rs6257 polymorphism, i.e., the subject's genotype is CC or CT. By increased susceptibility, it is meant that the subject's risk of developing type 2 diabetes may be predicted to be greater than the risk that a wild-type individual, i.e., TT for rs6257, has of developing type 2 diabetes. In certain embodiments, the increased risk may be predicted as being 140% the risk of that of the wild type individual. In other words, the subject may be predicted to be 1.4-fold more likely to develop type 2 diabetes than an individual that is wild type for rs6257. In other embodiments, the method includes predicting a decreased susceptibility to developing type 2 diabetes if the subject carries the A allele of the rs6259 polymorphism, i.e., the subject's genotype is AA or AG. By decreased susceptibility, it is meant that the subject's risk of developing type 2 diabetes may be predicted to be less than the risk that a wild-type individual, i.e., GG for rs6259, has of developing type 2 diabetes. In some embodiments, the decreased risk may be predicted as being about 66%, i.e., two-thirds, the risk of that of the wild type individual. In other words, the subject with the rs6259 polymorphism may be predicted to be 1.5-fold less likely (1/0.66=1.5) to develop type 2 diabetes than an individual that is wild type for rs6259.

In certain embodiments, the methods include genotyping a subject for more than one SHBG polymorphism, e.g., both the rs6257 and rs6259 polymorphisms, where the values of both genotypes are used to predict the susceptibility of the subject to developing type 2 diabetes. For example, the methods may include predicting a decreased susceptibility for developing type 2 diabetes if the subject is genotypically an A carrier for rs6259 (i.e., AG or AA) and wild-type for rs6257 (i.e., TT). In certain embodiments, the decreased risk may be predicted as being 40%, i.e., about four-tenths, the risk that an individual that is genotypically situated to have a low SHBG concentration, e.g., an individual that is genotypically wild-type for rs6259 (i.e. GG) and a C carrier for rs6257 (i.e., CT or CC), has of developing type 2 diabetes. In other words, the subject may be predicted to be about 2-fold less likely (1/0.43=2.3) to develop type 2 diabetes than the rs6259^{G/G}; rS6257^{CT or CC} individual. As another example, a decreased susceptibility for developing type 2 diabetes may be predicted if the subject is genotypically wild-type for rs6259 (i.e. GG) and wild type for rs6257 (i.e. TT). In certain embodiments, the decreased risk may be predicted as being about 60%, i.e. about six-tenths, the risk of that of the rs6259^{G/G}; rs6257^{CT or CC} individual; in other words, the subject may be predicted to be 1.6-fold less likely to develop type 2 diabetes than the rs6259^{G/G}; rs6257^{CT or CC} individual. As a third example, a decreased susceptibility for developing type 2 diabetes may be predicted if the subject is genotypically AG or AA for rs6259 (i.e., an A carrier) and CT or CC for rs6257. In certain embodiments, the decreased risk may be predicted as being 85%, or eight-tenths, OF CC tenths, the risk of that of the rs6259^{G/G}; individual; in other words, the subject may be predicted to be 1.3-fold less likely to develop type 2 diabetes than the rs6259^{G/G}; rs6257^{CT or CC} individual.

In some embodiments, the method includes predicting a susceptibility to developing type 2 diabetes based upon an SHBG concentration in a fluid sample. In certain embodiments, a decreased susceptibility to developing type 2 diabetes may be predicted if the subject has an SHBG concentration that is greater than 15 nmol/liter, 24 nmol/liter, 35 nmol/liter, 44 nmol/liter, 55 nmol/liter; such as greater than 17 nmol/liter, 24 nmol/liter, 35 nmol/liter, or 44 nmol/liter for women, and 15 nmol/liter, 19 nmol/liter, 26 nmol/liter, or 34 nmol/liter for men. In other words, subjects with an SHBG level this is higher than this reference value may be predicted to have a reduced/decreased likelihood of developing type 2 diabetes in comparison to individuals with an SHBG concentration of this reference value or lower, whereas a subject with an SHBG concentration that is about this reference value or lower may be predicted to have an equal likelihood as not of developing type 2 diabetes. In other words, subjects with an SHBG level that is higher than this reference value may be predicted to be less likely to become diabetic than individuals with SHBG levels that are about the reference value or lower.

For example, a woman with a SHBG concentration of 25 nmol/liter or greater may be predicted as having at most 16% the risk, that is, at most about one-sixth the risk, of developing type 2 diabetes as a woman with an SHBG concentration of 24 nmol/liter or less, e.g. of about 5.8-24.7 nmol/liter. In other words, the subject may be predicted to be 6-fold or more less likely (1/0.16=6.25) to develop type 2 diabetes than a woman with an SHBG concentration of about 24 nmol/liter or less. In some instances, a woman with an SHBG concentration of about 25-35 nmol/liter may be predicted as having 16% the risk, that is, about one-sixth the risk, of developing type 2 diabetes as a woman with an SHBG concentration of 24 nmol/liter or less, e.g., of about 5.8-24.7 nmol/liter. In other words, the subject may be predicted to be 6-fold or more less likely (1/0.16=6.25) to develop type 2 diabetes than a woman with an SHBG concentration of 24 nmol/liter or less. A woman with a SHBG concentration of 35-44 nmol/liter may be predicted as having 4% the risk, that is, one twenty-fifth the risk, of developing type 2 diabetes as a woman with a SHBG concentration of 24 nmol/liter or less, e.g., of 5.8-24.7 nmol/liter. In other words, the subject may be predicted to be 25-fold less likely (1/0.04=25) to develop type 2 diabetes than a woman with a SHBG concentration of 25 nmol/liter or less. A woman with a SHBG concentration of 44-122 nmol/liter may be predicted as having 9% the risk, that is, slightly less than one tenth the risk, of developing type 2 diabetes as a woman with a SHBG concentration of 24 nmol/liter or less, e.g. of about 5.8-24.7 nmol/liter. In other words, the subject may be predicted as being 11-fold less likely (1/0.09=11) to develop type 2 diabetes than a woman with a SHBG concentration of 24 nmol/liter or less.

Likewise, a man with a SHBG concentration of 20 nmol/liter or greater may be predicted as having at most 48% the risk, that is, slightly less than twice the risk, of developing type 2 diabetes as a man with a SHBG concentration of 19 nmol/liter or less, e.g., of about 4.4-19.4 nmol/liter. In other words, the male subject may be predicted to be at 2-fold or more less likely (1/0.48=2.1) to develop type 2 diabetes than a man with a SHBG concentration of 19 nmol/liter or less. In some instances, a man with a SHBG concentration of 20-25 nmol/liter may be predicted as having 48% the risk, that is, slightly less than twice the risk, of developing type 2 diabetes as a man with a SHBG concentration of 19 nmol/liter or less, e.g., of 4.4-19.4 nmol/liter. In other words, the subject may be predicted to be 2-fold less likely (1/0.48=2.1) to develop type 2 diabetes than a man with a SHBG concentration of 19 nmol/liter or less. A man with a SHBG concentration of 25-34 nmol/liter may be predicted as having 41% the risk, that is, slightly less than twice the risk, of developing type 2 diabetes as a man with a SHBG concentration of 19 nmol/liter or less, e.g. of about 4.4-19.4 nmol/liter. In other words, the subject may be predicted to be 2.5-fold less likely (1/0.41=2.4) to develop type 2 diabetes than a man with an SHBG concentration of 19 nmol/liter or less. A man with an SHBG concentration of 34-76 nmol/liter may be predicted as having about 10% the risk, that is, about one tenth the risk, of developing type 2 diabetes as a man with a SHBG concentration of 19 nmol/liter or less, e.g., of about 4.4-19.4 nmol/liter. In other words, the subject may be predicted as being 10-fold less likely (1/0.10=10) to develop type 2 diabetes than a man with a SHBG concentration of 19 nmol/liter or less.

Thus, the instant invention may be employed to readily predict not only if a subject has an increased or decreased susceptibility to developing type 2 diabetes, but also by how much this level of susceptibility is increased or decreased.

In certain embodiments, the methods further include evaluating the subject for the presence of one or more additional type 2 diabetes prediction factors, including Body Mass Index (BMI), smoking status, alcohol use, degree of exercise, history of hypertension, family history of diabetes, multivitamin use, past hormone-replacement therapy (in women), years of oral contraceptive use (in women), years since menopause (women), cause of menopause (women), waist circumference, amount of C-reactive protein (CRP), amount of glycated hemoglobin (e.g., HbA1c), testosterone levels, and estradiol levels, where any such factors may be employed. In some instances, a panel of biomarkers is obtained of which SHBG level is one of the markers, where the panel of two or more biomarkers is employed to predict susceptibility of a subject to developing type 2 diabetes. The predictive power of SHBG levels for predicting susceptibility of a subject to developing type 2 diabetes is sufficiently strong independent of these factors, but SHBG level values may be used in conjunction with these other risk factors to improve the positive predictive value (PPV) of risk predictions, that is, the strength or power of risk predictions, made with these other factors. By positive predictive value it is meant the probability of the disease being present if the test is positive, i.e. the number of truly susceptible patients identified as susceptible (i.e., true positives (TP)) divided by the total number of susceptible patients identified (i.e. the sum of true positives identified (TP) and false positives identified (FP)). In some embodiments, accounting for SHBG levels improves the PPV of predictions of a subject's susceptibility to and risk of developing type 2 diabetes that rely upon evaluating a subject for one or more other type 2 diabetes prediction factors by 2%, by 4%, by 7%, or by 10%. In other words, a method that includes both evaluating a subject for SHBG levels and evaluating a subject for the presence of one or more additional type 2 diabetes prediction factors provides for an improved positive predictive value (PPV) of 2%, 4%, 7% of 10% for the prediction over a method of predicting a subject's susceptibility to and risk of developing type 2 diabetes that relies upon evaluating a subject for one or more said additional type 2 diabetes prediction factors and that does not account for SHBG level values.

In some embodiments, the prediction of a subject's susceptibility to developing type 2 diabetes includes generating a written report that includes the artisan's prediction of the subject's susceptibility to and risk of developing type 2 diabetes, i.e. a “susceptibility prediction.” Thus, a subject method may further include a step of generating or outputting a report providing the results of a risk likelihood assessment, which report can be provided in the form of an electronic medium (e.g., an electronic display on a computer monitor), or in the form of a tangible medium (e.g., a report printed on paper or other tangible medium).

A “report,” as described herein, is an electronic or tangible document which includes report elements that provide information of interest relating to a subject likelihood assessment and its results. A subject report includes at least a susceptibility prediction, i.e. a prediction as to the susceptibility of a patient to developing type 2 diabetes. A subject report can be completely or partially electronically generated. A subject report can further include one or more of: 1) information regarding the testing facility; 2) service provider information; 3) patient data; 4) sample data; 5) an assessment report, which can include various information including: a) reference values employed, and b) test data, where test data can include: i) SHBG concentration; and/or ii) genotypes for one or more SHBG polymorphisms; 6) other features.

The report may include information about the testing facility, which information is relevant to the hospital, clinic, or laboratory in which sample gathering and/or data generation was conducted. Sample gathering can include obtaining a fluid sample, e.g. blood, saliva, urine etc.; a tissue sample, e.g. a tissue biopsy, a swab of the lining of the mouth or nose, a collect hair follicle, etc. from a subject. Data generation can include one or more of: a) measuring a level of SHBG polypeptide concentration; and b) genotyping for one or more SHBG polymorphism. This information can include one or more details relating to, for example, the name and location of the testing facility, the identity of the lab technician who conducted the assay and/or who entered the input data, the date and time the assay was conducted and/or analyzed, the location where the sample and/or result data is stored, the lot number of the reagents (e.g., kit, etc.) used in the assay, and the like. Report fields with this information can generally be populated using information provided by the user.

The report may include information about the service provider, which may be located outside the healthcare facility at which the user is located, or within the healthcare facility. Examples of such information can include the name and location of the service provider, the name of the reviewer, and where necessary or desired the name of the individual who conducted sample gathering and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The report may include a patient data section, including patient medical history (which can include, e.g., age, race, fasting status, Body Mass Index (BMI), smoking status, alcohol use, degree of exercise, history of hypertension, family history of diabetes, multivitamin use, past hormone-replacement therapy (in women), years of oral contraceptive use (in women), years since menopause (women), and cause of menopause (women), waist circumference), as well as administrative patient data (that is, data that are not essential to the susceptibility prediction) such as information to identify the patient (e.g., name, patient date of birth (DOB), gender, mailing and/or residence address, medical record number (MRN), room and/or bed number in a healthcare facility), insurance information, and the like), the name of the patient's physician or other health professional who ordered the susceptibility prediction and, if different from the ordering physician, the name of a staff physician who is responsible for the patient's care (e.g., primary care physician).

The report may include a sample data section, which may provide information about the biological sample analyzed in the susceptibility prediction, such as the source of biological sample obtained from the patient (e.g. blood, saliva, or type of tissue, etc.), how the sample was handled (e.g. storage temperature, preparatory protocols) and the date and time collected. Report fields with this information can generally be populated using data entered by the user, some of which may be provided as pre-scripted selections (e.g., using a drop-down menu).

The report may include an assessment report section, which may include information generated after processing of the data as described herein. The interpretive report can include a prediction of the likelihood that the subject will develop type 2 diabetes. The interpretive report can include, for example, result of genotyping for rs6257 SHBG polymorphism (e.g., “CC” or “CT”, or “C allele identified”); and/or results of genotyping for rs6257 SHBG polymorphism; and/or results of SHBG concentration assay (e.g., “37 nmol/liter”); and interpretation, i.e. prediction. The assessment portion of the report can optionally also include a Recommendation(s). For example, where the results indicate an increased susceptibility to and risk of developing type 2 diabetes, the recommendation can include a recommendation that diet or lifestyle be altered or medical intervention be provided as recommended in the art.

It will also be readily appreciated that the reports can include additional elements or modified elements. For example, where electronic, the report can contain hyperlinks which point to internal or external databases which provide more detailed information about selected elements of the report. For example, the patient data element of the report can include a hyperlink to an electronic patient record, or a site for accessing such a patient record, which patient record is maintained in a confidential database. This latter embodiment may be of interest in an in-hospital system or in-clinic setting. When in electronic format, the report is recorded on a suitable physical medium, such as a computer readable medium, e.g., in a computer memory, zip drive, CD, DVD, etc.

It will be readily appreciated that the report can include all or some of the elements above, with the proviso that the report generally includes at least the elements sufficient to provide the analysis requested by the user (e.g., susceptibility prediction).

The subject methods further find use in therapeutic applications. In these applications, a subject's susceptibility to and risk of developing for developing type 2 diabetes is first predicted. The subject is then treated using a pharmacological protocol, where the suitability of the protocol for a particular subject is determined using the results of the prediction step. By “treatment” is meant that at least an amelioration of the symptoms associated with the condition afflicting the subject is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g. prevented from happening, or stopped, e.g. terminated, such that the subject no longer suffers from the condition, or at least the symptoms that characterize the condition. For example, where the subject is predicted to be susceptible to or have a risk of developing type 2 diabetes, a preventative therapeutic regimen may be prescribed for the subject. A suitable regimen is any regimen that is known in the art to be preventative for type 2 diabetes, for example, a regimen comprising the prescription diabetes drug Metformin (N,N-dimethylimidodicarbonimidic diamide) e.g., sold under the tradenames Glucophage, Glucophage XR, Riomet, Fortamet, Glumetza, Obimet, Dianben, Diabex, Diaformin, the weight loss drug Orlistat ([(1S)-1-[(2S,3S)-3-hexyl-4-oxo-oxetan-2-yl]methyl]dodecyl](2S)-2-formamido-4-methyl-pentanoate) e.g., sold under the tradename Xenical, or the diabetes medication Acarbose ((2R,3R,4R,5S,6R)-5-{[(2R,3R,4R,5S,6R)-5-{[(2R,3R,4S,5S,6R)-3,4-dihydroxy-6-methyl-5-{[(1S,4R,5S,6S)-4,5,6-trihydroxy-3-(hydroxymethyl)cyclohex-2-en-1-yl]amino}tetrahydro-2H-pyran-2-yl]oxy}-3,4-dihydroxy-6-(hydroxymethyl)tetrahydro-2H-pyran-2-yl]oxy}-6-(hydroxymethyl)tetrahydro-2H-pyran-2,3,4-triol), e.g., sold under the tradenames Precose, Glucobay, Prandase, etc. Alternatively and/or in addition, the subject may be instructed to adopt one or more lifestyle modifications. For example, the subject may be counseled to perform regular exercise, e.g., 30 minutes a day, five days a week, and/or to partake of a modified diet, e.g., one that is low in fat and high in fiber.

In addition or alternatively from predicting susceptibility to and risk of type 2 diabetes, embodiments of the methods may find use in predicting increased susceptibility in developing diabetes-related complications, such as neuropathy, resulting in tingling, numbness, burning, pain, or loss of all sensation in the affected limbs, as well as erectile dysfunction and problems with digestion such as nausea, vomiting, diarrhea or constipation; blindness; nephropathy; heart and blood vessel disease, e.g. coronary artery disease with chest pain (angina), heart attack, stroke, narrowing of the arteries (atherosclerosis) and high blood pressure; skin and mouth conditions, including bacterial and fungal infections; osteoporosis; and Alzheimer's disease. Methods of predicting increased or decreased susceptibility to and risk of developing these complications generally mirror those methods for predicting susceptibility to and risk of developing type 2 diabetes that rely upon obtaining an SHBG level value as described above.

Also of interest is the use of SHBG plasma values as a measure of the efficacy of type 2 diabetes therapies. For example, by assessing SHBG plasma value levels, one can determine whether a given therapy is working or not, that is, whether a given therapy is preventing the onset or progression of type 2 diabetes and/or complications associated with type 2 diabetes. In some embodiments, an increase in SHBG levels indicates that the type 2 diabetes therapy is effective, whereas no change or a decrease in SHBG levels indicates that a type 2 diabetes therapy is ineffective.

Also provided are methods of inhibiting the development of type 2 diabetes in a subject, and for treating a subject for type 2 diabetes. In embodiments of these methods, an effective amount of agent that increases the amount or activity of circulating SHBG concentration in the subject e.g., by exogenously contributing to the pool of circulating SHGB polypeptide, by enhancing the endogenous SHBG output from the liver, by increasing the affinity or stability of the interaction between SHBG and testosterone or estradiol, etc., is administered to the subject to treat the subject for type 2 diabetes. In some embodiments, the agent is a peptide agent. In certain embodiments, the peptide agent is an SHBG polypeptide, or fragment thereof. SHBG polypeptides may comprise amino acid sequence of SHBG variant/isoform 1 (SEQ ID NO:2), variant/isoform 2 (SEQ ID NO:4); variant/isoform 3 (SEQ ID NO:6); or variant/isoform 4 (SEQ ID NO:8). The SHBG sequence may be from any mammalian or avian species, e.g. primate sp., particularly humans; rodents, including mice, rats and hamsters; rabbits; equines, bovines, canines, felines; etc. Of particular interest are the human proteins. Of particular interest in some embodiments are SHBG polypeptide fragments that bind to testosterone and estradiol.

In some embodiments, an SHBG polypeptide, or a functional fragment thereof is administered to a patient. SHBG polypeptides useful in this invention also include derivatives, variants, and biologically active fragments of naturally occurring SHBG polypeptides, and the like. A “variant” polypeptide means a biologically active polypeptide having less than 100% sequence identity with a native sequence polypeptide. Such variants include polypeptides wherein one or more amino acid residues are added at the N- or C-terminus of, or within, the native sequence; from about one to forty amino acid residues are deleted, and optionally substituted by one or more amino acid residues; and derivatives of the above polypeptides, wherein an amino acid residue has been covalently modified so that the resulting product has a non-naturally occurring amino acid. Ordinarily, a biologically active variant will have an amino acid sequence having at least about 80% amino acid sequence identity with a native sequence polypeptide, at least about 90% sequence identity, about 95% sequence identity, about 99% sequence identity, or will be substantially the same as or identical to the native sequence polypeptide.

The sequence of SHBG polypeptides as described above may be altered in various ways known in the art to generate targeted changes in sequence. The sequence changes may be substitutions, insertions or deletions. Such alterations may be used to alter properties of the protein, by affecting the stability, specificity, etc. Techniques for in vitro mutagenesis of cloned genes are known. Examples of protocols for scanning mutations may be found in Gustin et al., Biotechniques 14:22 (1993); Barany, Gene 37:111-23 (1985); Colicelli et al., Mol Gen Genet. 199:537-9 (1985); and Prentki et al., Gene 29:303-13 (1984). Methods for site specific mutagenesis can be found in Sambrook et al., Molecular Cloning: A Laboratory Manual, CSH Press 1989, pp. 15.3-15.108; Weiner et al., Gene 126:35-41 (1993); Sayers et al., Biotechniques 13:592-6 (1992); Jones and Winistorfer, Biotechniques 12:528-30 (1992); Barton et al., Nucleic Acids Res 18:7349-55 (1990); Marotti and Tomich, Gene Anal Tech 6:67-70 (1989); and Zhu Anal Biochem 177:120-4 (1989).

The peptides may be joined to a wide variety of other oligopeptides or proteins for a variety of purposes. By providing for expression of the subject peptides, various post-expression modifications may be achieved. For example, the peptides may be PEGylated, where the polyethyleneoxy group provides for enhanced lifetime in the blood stream. The SHBG polypeptide may be fused to another polypeptide to provide for added functionality, e.g. to increase the in vivo stability. Generally such fusion partners are a stable plasma protein, which may, for example, extend the in vivo plasma half-life of the SHBG polypeptide when present as a fusion, in particular wherein such a stable plasma protein is an immunoglobulin constant domain.

In cases where the stable plasma protein is normally found in a multimeric form, e.g., immunoglobulins or lipoproteins, in which the same or different polypeptide chains are normally disulfide and/or noncovalently bound to form an assembled multichain polypeptide, the fusions herein containing the SHBG polypeptide may also be produced and employed as a multimer having substantially the same structure as the stable plasma protein precursor. These multimers may be homogeneous with respect to the SHBG they comprise, or they may contain more than one SHBG polypeptide.

Stable plasma proteins may have from 30 to 2,000 residues, which exhibit in their native environment an extended half-life in the circulation, e.g., greater than about 20 hours. Examples of suitable stable plasma proteins are immunoglobulins, albumin, lipoproteins, apolipoproteins and transferrin. The SHBG polypeptide may be fused to a plasma protein, e.g., IgG at the N-terminus of the plasma protein or fragment thereof, which is capable of conferring an extended half-life upon the SHBG polypeptide.

The SHBG polypeptide for use in the subject methods may be produced from eukaryotic or prokaryotic cells, or may be synthesized in vitro. Where the protein is produced by prokaryotic cells, it may be further processed by unfolding, e.g. heat denaturation, DTT reduction, etc. and may be further refolded, using methods known in the art.

Modifications of interest that do not alter primary sequence include chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also included in the subject invention are polypeptides that have been modified using ordinary molecular biological techniques and synthetic chemistry so as to improve their resistance to proteolytic degradation or to optimize solubility properties or to render them more suitable as a therapeutic agent. Analogs of such polypeptides include those containing residues other than naturally occurring L-amino acids, e.g. D-amino acids or non-naturally occurring synthetic amino acids. D-amino acids may be substituted for some or all of the amino acid residues.

The subject polypeptides may be prepared by in vitro synthesis, using conventional methods as known in the art. Various commercial synthetic apparatuses are available, for example, automated synthesizers by Applied Biosystems, Inc., Foster City, Calif., Beckman, etc. By using synthesizers, naturally occurring amino acids may be substituted with unnatural amino acids. The particular sequence and the manner of preparation will be determined by convenience, economics, purity required, and the like.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

In one embodiment of the invention, the SHBG polypeptide consists essentially of a polypeptide sequence of at least 476 amino acids in length and having a sequence of an SHBG polypeptide as described in NM_{—}001040. By “consisting essentially of” in the context of a polypeptide described herein, it is meant that the polypeptide is composed of the SHBG polypeptide sequence, which sequence is optionally flanked by one or more amino acid or other residues that do not materially affect the basic characteristic(s) of the polypeptide.

In certain embodiments, the agent is a nucleic acid agent, e.g., an SHBG coding sequence which increases expression of SHBG. The nucleic acid sequences encoding the above SHBG polypeptides may be accessed from public databases. Identification of additional SHBG polypeptide variants is accomplished by conventional screening methods of DNA libraries or biological samples for DNA sequences having a high degree of similarity to known SHBG polypeptides. Polynucleotides of interest include those that encode a polypeptide that consists essentially of a polypeptide sequence of at least about 50 amino acids, usually at least about 100 amino acids, at least about 150 amino acids, at least about 200 amino acids, at least about 250 amino acids, at least about 300 amino acids and which may include up to the full length of amino acids of an SHBG polypeptide variant. Such polynucleotides may be operably joined to control sequences, e.g. for transcriptional start, stop, translation, promoters, etc. Polynucleotides may also include an SHBG coding sequence combined with fusion polypeptide sequences.

SHBG coding sequences can be generated by methods known in the art, e.g., by in vitro synthesis, recombinant methods, etc. to provide a coding sequence to corresponds to an SHBG polypeptide. Using the known genetic code, one can produce a suitable coding sequence. Double or single stranded fragments can be obtained from the DNA sequence by chemically synthesizing oligonucleotides in accordance with conventional methods, by restriction enzyme digestion, by PCR amplification, etc.

SHBG encoding nucleic acids can be provided as a linear molecule or within a circular molecule, and can be provided within autonomously replicating molecules (vectors) or within molecules without replication sequences, e.g., minicircies. Expression of the nucleic acids can be regulated by their own or by other regulatory sequences known in the art. The nucleic acids can be introduced into suitable host cells using a variety of techniques available in the art, such as transferrin polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

In certain embodiments, the agent is a small molecule, e.g., a small organic compound, e.g., a compound that is 5000 daltons or less. Naturally occurring or synthetic small molecule compounds of interest include numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Such molecules may be identified, among other ways, by employing the screening protocols described below.

In any of the above methods, the subject may vary. In certain embodiments, the subjects are “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g., rabbits), and primates (e.g., humans, chimpanzees, and monkeys). In certain embodiments, the subjects are humans.

### Reagents, Devices and Kits

Also provided are reagents, devices and kits thereof for practicing one or more of the above-described methods. The subject reagents, devices and kits thereof may vary greatly. Reagents and devices of interest include those mentioned above with respect to the methods of identifying the presence of the target polymorphisms, where such reagents may include nucleic acid primers, arrays of nucleic acid probes, antibodies to polymorphic polypeptides (e.g., immobilized on a substrate), signal producing system reagents, etc., depending on the particular detection protocol to be performed.

In some instances, devices of the invention are those configured to obtain a sex hormone-binding globulin (SHBG) level value for a sample from a subject; and then output a prediction of the subject's susceptibility to and risk of developing type 2 diabetes based on the obtained sex hormone-binding globulin (SHBG) level value. Such devices may include a one or more different types of reagents, e.g., depending on the particular assay that is to be conducted with the device. For example, the devices may include a ligand that specifically binds to SHBG, such as an SHBG antibody or binding fragment thereof. Alternatively, the device may be configured to genotype a subject for a SHBG polymorphism. Such devices may include an SHBG polymorph detection reagent, such as one configured to detect one or more of the rs6257 and rs6259 SHBG polymorphisms. Also of interest are devices that are configured to obtain a sex hormone-binding globulin (SHBG) level value for a sample from a subject, wherein said devices comprise a ligand that specifically binds to SHBG; and an SHBG polymorphism detection element. Examples of specific reagent and device formats of interest are described in greater detail above.

For kits, the various components of the kit may be present in separate containers or certain compatible components may be pre-combined into a single container, as desired.

In addition to the above components, the kits of the invention may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The following examples are offered by way of illustration and not by way of limitation.

### Experimental

We investigated the relationship between levels of sex hormone-binding globulin (SHGB) and SHBG polymorphisms with the risk of type 2 diabetes in a prospective study of postmenopausal women. SHBG germ-line variants that were found to be predictive of both the risk of type 2 diabetes and the level of sex hormone-binding globulin were further evaluated to the association between levels of sex hormone-binding globulin and the risk of type 2 diabetes by using mendelian randomization with SHBG genotypes as instruments to minimize residual confounding and reverse causation (since genotypes are thought to be independent of confounders and not to be modified by disease processes). To confirm relations between SHBG genotypes, levels of SHBG, and risk of type 2 diabetes, we conducted replication analyses in an independent cohort of men.

### Methods

Study Population. The Women's Health Study, begun in 1993, is a randomized, double-blind, placebo-controlled, 2-by-2 factorial study of low-dose aspirin and vitamin E for the primary prevention of cardiovascular disease and cancer in 39,876 female health professionals in the United States who, at enrollment, were 45 years of age or older and did not have diabetes, cancer (other than nonmelanoma skin cancer), or cardiovascular disease (Liu S, et al. (2006) Diabetes 55:2856-2862). A total of 28,345 women provided blood samples at baseline; 12,304 were postmenopausal and were not using hormone-replacement therapy at the time of blood collection. These 12,304 women were included in our study because hormone-replacement therapy influences levels of sex hormones and sex hormone-binding globulin. During a 10-year follow-up period, we identified 366 cases of newly diagnosed type 2 diabetes. Using risk-set sampling (Wacholder S, et al. (1992) Am J Epidemiol 135:1019-1028), we randomly selected controls from among women who remained free from type 2 diabetes and matched them to case patients, in a 1:1 ratio, according to age (within 1 year), duration of the follow-up period (within 1 month), self-reported race, and fasting status at the time of blood draw (with 72% of patients fasting, defined as there having been at least 10 hours since the previous meal). On the basis of these criteria, 359 case patients and 359 matched controls were selected from the Women's Health Study cohort.

A replication study was conducted within the Physicians' Health Study II (PHS II) of men (Sesso H D, et al. (2008) JAMA 300:2123-2133) This study is a randomized, double-blind, placebo-controlled 2×2×2×2 factorial trial of vitamin E, vitamin C, β-carotene, and a multivitamin in the prevention of CVD and cancer among 14,641 US male physicians aged 50 years and older free of baseline cancer and CVD (Christensen L, et al. (1997) Dan Med Bull 44(5):547-50). The PHS II is an extension of the Physicians' Health Study I (PHS I) (Hennekens C H, et al. (1996) N Engl J Med 334(18):1145-9; (1989) N Engl J Med 321(3):129-35), a 2×2 factorial trial of low-dose aspirin and β-carotene among 22,071 healthy male physicians aged 40-84 at entry in 1982. Recruitment for PHS II was completed in two phases. In the first phase, which began in July 1997, PHS I participants were invited to enroll in PHS II. In the second phase, which took place from July 1999 to July 2001, new participants were recruited from a roster of physicians provided by the American Medical Association. Of the 14,641 Physicians' Health Study II participants, 11,130 provided blood samples at baseline. During 8 years of follow-up, diabetes developed in 170 initially healthy men. Risk-set sampling identical to that used in the Women's Health Study was applied to prospectively selected controls from the cohort person-time. Controls were randomly selected to match cases, in a 1:1 ratio, according to age (within 1 year), duration of the follow-up period (within 1 month), self-reported race, and time of blood draw. On the basis of these criteria, 170 cases and 170 controls were selected.

All participants in the Women's Health Study and the Physicians' Health Study II provided written informed consent before enrollment. This study was approved by the research review boards of Partners HealthCare and the University of California at Los Angeles (UCLA).

Laboratory Procedures. Plasma samples were stored in liquid nitrogen tanks until analysis. Matched case and control specimens were handled identically and were assayed in random order within each pair in the same analytical run for each cohort. Laboratory personnel were unaware of the case-control status during all assays. Levels of sex hormone-binding globulin were measured with the use of a chemiluminescent immunoassay (with an Elecsys 2010 autoanalyzer, Roche Diagnostics), validated for plasma sex hormone-binding globulin (Reynders M, et al. (2005) Clin Chem Lab Med 43:86-89). The coefficient of variation for sex hormone-binding globulin data among blinded quality-control samples was 2.8%. Genotyping of SHBG polymorphisms of women was conducted at the Harvard Cancer Center's High-Throughput Polymorphism Detection Core laboratory. Replication genotyping in men was conducted through the Program on Genomics and Nutrition at UCLA. Overall, five SNPs were genotyped in women (with a success rate of ≧95%). Three noninformative SNPs were excluded from analysis: the rs6260 and rs9282845 loci had a minor-allele frequency of 0%, and rs6258 had a minor-allele frequency of less than 1%. Two informative SNPs, rs6257 and rs6259, were included in our study and were included for genotyping in the replication study (with a success rate of >99%). The frequencies of the genotypes were found to be consistent with Hardy-Weinberg equilibrium among controls (P>0.05 for all comparisons).

Genotyping. To identify common genetic variants across the SHBG gene that have known functional consequences, we initially surveyed all the common SNPs spanning 4 kb of the SHBG gene, covering at least its 10 kb 5′ upstream regions. This initial assessment was based on extensive sequencing done the National Cancer Institute's cohort consortium, located on the world wide web at the address uscnorriscancer.usc.edu/Core/DocManager/DocumentList.aspx. We further searched the National Center for Biotechnology Information database SNP (NCBI dbSNP) for additional functional SNPs. The set of 5 putative functional SNPs with a minor allele frequency (MAF) ≧5% in at least one ethnic group were genotyped in our samples. All DNA samples were genotyped using the ABI PRISM 7900HT Sequence Detection System (Applied Biosystems, Foster City, Calif.), in 384-well format. 5-nuclease assay (TaqMan) was used to distinguish gene alleles. PCR amplification was carried out on 5-20 ng DNA using 1×TaqMan universal PCR master mix (No Amp-erase UNG), 900 nM forward and reverse primers, 200 nM of the FAM labeled probe and 200 nM of the VIC labeled probe in 5 ml reaction volume. Amplification conditions on AB 7900 dual plate thermal cycle (Applied Biosystems, Foster City, Calif.) were as follows: 1 cycle of 95° C. for 10 min, followed by 50 cycles of 92° C. for 15s and 60° C. for 1 min. Although the rs6257 in intron 1, sited 17 bp upstream of exon 2, is significantly associated with plasma SHBG levels, the precise mechanisms remain unknown. This intronic polymorphism may regulate SHBG expression through its location in a regulatory element or its high linkage disequilibrium (LD) with an as yet unknown functional variant located in the promoter region. Rs6259, located within exon 8, is a nonsynonymous amino acid substitution of asparagines (Asn) for aspartic acid (Asp) and introduces an additional N-glycosylation consensus site which may lead to reduced clearance rate of SHBG from the circulation by altering the binding of SHBG to membrane receptors or other proteins.

TABLE 1
Descriptive characteristics of Sex Hormone-Binding Globulin polymorphisms genotyped.

| | | | | | Ammo Acid | Hydropathy | Acid/Base |
|---|---|---|---|---|---|---|---|
| Gene | SNP ID | Gene Region | Allele* | Function | Substitution | Index^{†} | Property |
| SHBG | rs6257 | Intron | [C/T] | — | — | — | — |
| | | 1, −17 bp Exon 2 | | | | | |
| SHBG | rs6258 | Exon 4 | [T/C] | Non-syn | Leu for Pro | 3.8, 1.6 | Neutral, |
| | | | | | | | neutral |
| SHBG | rs6259 | Exon 8 | [A/G] | Non-syn | Asn for Asp | −3.5, −3.5 | Neutral, acidic |
| SHBG | rs6260 | Exon 1 | [G/A] | Non-syn | His for Arg | −3.2, −3.5 | Weak base, |
| SHBG | rs9282845 | Exon 1 | [A/G] | Non-syn | His for Arg | | strong base |

*Allele order designates [variant/ancestral] alleles
^{†}Hydropathy Index⁷ describes the hydrophobic (a.k.a. lipophilic) nature of amino acids; larger values indicate greater hydrophobicity

Statistical Analysis. Baseline characteristics were compared between case patients and controls using mixed-effects regression analysis for clustered data and conditional logistic-regression analysis. We divided the distributions of levels of sex hormone-binding globulin among controls into quartiles and compared baseline characteristics across the quartiles. Because the incidence-density sampling method was used to match controls to case patients on the basis of the cohort person-time (Wacholder S, et al. (1992) Am J Epidemiol 135:1019-1028), odds ratios (and 95% confidence intervals) for type 2 diabetes were computed by means of conditional logistic-regression analysis. Trend tests were computed to study the relations across increasing quartiles. In the primary multivariable model (“multivariable model 2” of Table 3), we adjusted for the factors used in matching controls and case patients, as well as the body-mass index (BMI, treated as a continuous variable), smoking (current, former, never), alcohol consumption (rarely/never, 1-3 drinks/month, 1-6 drinks/week, ≧1 drink/day), degree of exercise (rarely/never, 1, 2-3, 4-6 and ≧7 times/week), presence or absence of family history of diabetes (yes, no), history of hypertension (yes, no), past hormone-replacement therapy (HRT) use (yes, no), years of oral contraceptive use (<0.5, 0.5-2, >2 yrs), years of multivitamin use (current, former, never), years since menopause, and cause of menopause (natural vs. surgical, radiation, or chemotherapy). To assess confounding by reproductive and sociologic covariates in a sensitivity analysis, we fit an expanded model (“sensitivity model” of Table 3) to further adjust for age at menarche (<12, 12, 13, >13), total pregnancies (0, 1-2, 3-4, ≧5), pregnancies lasting months, age at first pregnancy of ≧6 months (none, <25, ≧25), marital status (current, former, never married), and education (high school, associates, bachelors, masters, doctoral). Similar analyses were carried out in men, with adjustment for BMI (continuous), smoking (current, former, never), alcohol consumption (drinks: <1/mo, 1-3/mo, 1/wk, 2-4/wk, 5-6/wk, 1/day, 2/day), exercise (0, 1, 2, 3, N4 days/wk), systolic blood pressure (continuous), current use of multivitamins, and family history of diabetes (yes, no).

Further sensitivity analyses excluded data for case patients in whom type 2 diabetes developed during the first 3 years of the follow-up period and accounted for waist circumference and baseline C-reactive protein (CRP) and glycated hemoglobin values. Owing to the apparent linearity of the observed associations and for parsimony, we expressed the odds ratio per natural-log standard-deviation increase when assessing the effect modification of BMI, past use of hormone-replacement therapy, years since menopause, and history of hypercholesterolemia or family history of diabetes. Geometric means and relative mean differences of levels of sex hormone-binding globulin between genotypes were calculated using linear regression analysis. Results between the sexes were pooled in random-effects models.

We used generalized linear models with instrumental variables (Hardin J W, et al. (2003) State J 3:351-60) to fit the data to regression models for levels of sex hormone-binding globulin and logistic-regression models for type 2 diabetes using SHBG germ-line variants as randomized instruments. Finally, prediction analyses were conducted with the use of receiver-operating-characteristic curves and C statistics to assess the relative predictive ability of sex hormone-binding globulin beyond that of traditional risk factors. All analyses were conducted using Stata software, version 9.2.

To further assess the independent utility of plasma SHBG levels for clinical prediction of Type 2 diabetes, we modeled SHBG while simultaneously adjusting for traditional Type 2 diabetes risk factors, CRP, and HbA1c, among those with normal HbA1c<6%. A C-statistic (are under a Receiver-Operator-Characteristic [ROC] curve) corresponding to a random non-informative predictor model has a value of 0.50, represented by the ROC curve as a diagonal line directly connecting coordinates (0,0) to (1,1). A C-statistic corresponding to a perfect predictor model has a value of 1.00, represented by lines connecting coordinates (0,0) to (0,1) and (0,1) to (1,1). Differences of C-statistics were used to compare models.

### Results

As expected, women in whom type 2 diabetes developed (case patients) generally had more adverse risk profiles at baseline than those who remained free of the disease (controls) (Table 2). Cross-sectional analyses at baseline revealed that higher levels of sex hormone-binding globulin were associated with lower BMI, a lower likelihood of having a history of hypertension, and more favorable lipid-profile and CRP levels (Table 3). Elevated levels of sex hormone-binding globulin were strongly and consistently associated with a reduced risk of type 2 diabetes (P for trend, <0.001) in both simple and multivariable analyses. Regardless of adjustment for a wide range of covariates or analysis through multiple sensitivity analyses, these findings did not materially change in direction or magnitude. The odds ratios of type 2 diabetes for quartiles 2, 3, and 4 (highest) of the sex hormone-binding globulin level, as compared with quartile 1 (lowest) were 1.00, 0.16 (95% confidence interval [CI], 0.08 to 0.33), 0.04 (95% CI, 0.01 to 0.12), and 0.09 (95% CI, 0.03 to 0.21), respectively (P value for trend, <0.001) (Table 3). In our independent replication study in men, results corroborated the strong inverse association between sex hormone-binding globulin levels and risk of type 2 diabetes (odds ratio for the highest vs. the lowest quartile, 0.10; 95% CI, 0.03 to 0.36); these findings remained highly robust in multiple sensitivity analyses (Table 4) and were consistent across subgroups (Table 5).

TABLE 2
Baseline Characteristics of Case Patients and Controls.

| Characteristic | Case Patients | | Controls | | P Value† |
|---|---|---|---|---|---|
| Women | | | | | |
| No. of participants | 359 | | 359 | | |
| Age-yr | 60.3 ± 6.1 | | 60.3 ± 6.1 | | |
| White race-%‡ | 92.5 | | 92.5 | | |
| Body-mass index§ | 30.9 ± 61 | | 26.0 ± 4.9 | | <0.001 |
| Alcohol use-g/day | 2.62 ± 7.4 | | 4.19 ± 8.3 | | 0.007 |
| Current smoking-% | 14.2 | | 13.7 | | 0.8.3 |
| Strenuous physical | 30.7 | | 38.7 | | 0.06 |
| activity ≧ once/wk-% | | | | | |
| Past postmenopausal | 32.0 | | 27.9 | | 0.22 |
| hormone use-% | | | | | |
| Any oral contraceptive | 49.9 | | 47.1 | | 0.23 |
| use-% | | | | | |
| Age at menopause-yr | 48.0 ± 6.1 | | 48.0 ± 5.7 | | 0.92 |
| Time since | 12.3 ± 8.2 | | 12.1 ± 7.9 | | 0.88 |
| menopause-yr | | | | | |
| Natural cause of | 63.0 | | 69.4 | | 0.08 |
| menopause-% | | | | | |
| Age at menarche, | 25.4 | | 21.7 | | 0.46 |
| <12 yr-% | | | | | |
| Age at first pregnancy of | 56.8 | | 48.8 | | 0.05 |
| ≧6-mo gestation, | | | | | |
| <25 yr-% | | | | | |
| Family history of | 48.5 | | 24.0 | | <0.001 |
| diabetes-% | | | | | |
| History of hypertension-% | 50.1 | | 30.4 | | <0.001 |
| ≧5 Pregnancies-% | 30.1 | | 34.0 | | 0.10 |
| Currently married-% | 63.5 | | 65.2 | | 0.15 |
| College graduate-% | 30.4 | | 38.7 | | 0.05 |
| SHBG-nmol/liter | 22.3 ± 13.8 | | 36.9 ± 17.4 | | <0.001 |
| SHBG SN P-no./total no. | | | | | |
| with data (%) | | | | | |
| rs6257 | | | | | 0.08 |
| Variant allele C | 76/337 | (22.6) | 65/344 | (18.9) | |
| Wild-type allele T | 261/337 | (77.4) | 279/344 | (81.1) | |
| rs6258 | | | | | 0.91 |
| Variant allele T | 2/339 | (0.6) | 4/347 | (1.2) | |
| Wild-type allele C | 337/339 | (99.4) | 343/347 | (98.8) | |
| rs6259 | | | | | 0.33 |
| Variant allele A | 66/342 | (19.3) | 82/337 | (24.3) | |
| Wild-type allele G | 276/342 | (80.7) | 255/337 | (75.7) | |
| Men | | | | | |
| No of participants | 170 | | 170 | | |
| Age-yr | 63.7 ± 7.6 | | 63.7 ± 7.6 | | |
| White race-%‡ | 85.3 | | 85.3 | | |
| Body-mass index§ | 28.9 ± 3.9 | | 25.5 ± 3.4 | | <0.001 |
| Alcohol use of ≧1 | 61.2 | | 62.4 | | 0.16 |
| drink/wk-% | | | | | |
| Current smoking-% | 6.5 | | 1.2 | | 0.06 |
| Vigorous physical | 55.3 | | 65.9 | | 0.34 |
| activity ≧1 day/wk-% | | | | | |
| Current multivitamin | 27.7 | | 27.1 | | 0.90 |
| use-% | | | | | |
| Systolic blood pressure- | 133 ± 14 | | 127 ± 11 | | <0.001 |
| mm Hg | | | | | |
| Hyperlipidemia-% | 67.1 | | 58.8 | | 0.09 |
| Family history of | 32.9 | | 17.1 | | 0.05 |
| diabetes-% | | | | | |
| SHBG-nmol/liter | 19.6 ± 7.2 | | 27.3 ± 10.7 | | <0.001 |
| SHBG SNP-no./total no. | | | | | |
| with data (%) | | | | | |
| rs6257 | | | | | 0.64 |
| Variant allele C | 33/167 | (19.8) | 29/164 | (17.7) | |
| Wild-type allele T | 34/167 | (80.2) | 35/164 | (82.3) | |
| rs6259 | | | | | 0.98 |
| Variant allele A | 27/167 | (16.2) | 41/163 | (25.2) | |
| Wild-type allele G | 140/167 | (83.8) | 122/163 | (74.8) | |

*Plus-minus values are means ± SD. SHBG denotes sex hormone-binding globulin, and SNP single-nucleotide polymor-phism.
†P values for continuous variables were calculated by means of mixed-effects models used to determine the mean difference between case patients and controls; for categorical variables, by means of tests for homogeneity across levels, from conditional logistic-regression analysis; and for SNPs, by means of tests of Hardy-Weinberg equilibrium among controls. P values are not shown for the variables used to match case patients and controls (age and race).
‡Race was self-reported.
§The body-mass index is the weight in kilograms divided by the square of the height in meters.

TABLE 3
Baseline Characteristics of Female Controls, According to
Sex Hormone-Binding Globulin (SHBG) Level.

| | SHBG Quartile | | | | |
|---|---|---|---|---|---|
| Characteristic | 1 (lowest) | 2 | 3 | 4 (highest) | P Value |
| SHBG (nmol/liter) | | | | | |
| Median | 17.1 | 29.3 | 39.0 | 55.8 | |
| Range | 5.8-24.7 | 24.8-34.6 | 34.7-44.3 | 44.4-122.4 | |
| Median age (yr) | 60.4 | 60.0 | 59.5 | 61.7 | 0.12 |
| Median time since menopause (yr) | 10.1 | 9.3 | 10.1 | 13.3 | 0.24 |
| Age at menarche, <12 yr (%) | 25.3 | 12.5 | 23.6 | 24.7 | 0.93 |
| Median body-mass index† | 28.3 | 26.2 | 24.2 | 23.7 | <0.001 |
| Median alcohol use (g/day) | 0 | 0.4 | 0.9 | 0.9 | 0.91 |
| Current smoking (%) | 12.1 | 6.8 | 20.2 | 14.6 | 0.20 |
| Physical activity ≧ once/wk (%) | 34.1 | 43.2 | 33.7 | 44.9 | 0.31 |
| Past postmenopausal hormone use (%) | 27.5 | 23.9 | 31.5 | 28.1 | 0.66 |
| Pregnancies, ≧5 (%) | 30.8 | 33.0 | 37.1 | 36.0 | 0.49 |
| Natural cause of menopause (%) | 70.3 | 69.3 | 61.8 | 76.4 | 0.63 |
| History of hypertension (%) | 47.2 | 28.4 | 20.2 | 25.8 | 0.001 |
| Family history of diabetes (%) | 27.5 | 23.9 | 19.1 | 25.8 | 0.63 |
| Median fasting LDL cholesterol (mg/dl) | 143 | 140 | 129 | 128 | <0.001 |
| Median fasting HDL cholesterol (mg/dl) | 47 | 49 | 53 | 57 | <0.001 |
| Median fasting triglycerides (mg/dl) | 150 | 118 | 82 | 88 | <0.001 |
| Median C-reactive protein (mg/liter) | 3.1 | 1.6 | 1.5 | 1.0 | <0.001 |
| Median glycated hemoglobin (%) | 5.13 | 5.06 | 5.02 | 5.09 | 0.12 |

*HDL denotes high-density lipoprotein, and LDL low-density lipoprotein. To convert the values for cholesterol to millimoles per liter, multiply by 0.02586. To convert the values for triglycerides to millimoles per liter, multiply by 0.01129.
†The body-mass index is the weight in kilograms divided by the square of the height in meters.

TABLE 4
Risk of Type 2 diabetes among Women and Men, According to Sex Hormone-Binding Globulin (SHBG) Level.

| | SHGB Quartile | | | | P Value |
|---|---|---|---|---|---|
| Variable | 1 (lowest) | 2 | 3 | 4 (highest) | for Trend |
| Woman | | | | | |
| Median SHBG level-nmol/liter (range) | 17.1 (5.8-24.7) | 29.3 (24.8-34.6) | 39.0 (34.7-44.3) | 55.8 (44.4-122) | |
| No. of participants- case patients/controls† | 267/91 | 49/88 | 19/89 | 24/89 | |
| Simple model 1-odds ratio (95% CI) | 1.00 | 0.26 (0.11-0.33) | 0.08 (0.04-0.16) | 0.12 (0.05-0.25) | <0.001 |
| Multivariable model 2-odds ratio (95% CI) | 1.00 | 0.16 (0.08-0.33) | 0.04 (0.01-0.12) | 0.09 (0.03-0.21) | <0.001 |
| Sensitivity model-odds ratio (95% CI) | | | | | |
| Multivariable + reproductive and sociologic | 1.00 | 0.11 (0.05-024) | 0.03 (0.01-0.10) | 0.08 (0.02-0.27) | <0.001 |
| covariates | | | | | |
| Multivariable + waist circumference | 1.00 | 0.16 (0.07-0.34) | 0.04 (0.01-0.12) | 0.09 (0.03-0.23) | <0.001 |
| Multivariable + C-reactive protein | 1.00 | 0.18 (0.08-0.39) | 0.05 (0.01-0.14) | 0.11 (0.04-0.27) | <0.001 |
| Multivariable + fasting LDL and HDL | 1.00 | 0.22 (0.09-0.51) | 0.06 (0.02-0.24) | 0.16 (0.06-0.45) | <0.001 |
| cholesterol and triglycerides | | | | | |
| Multivariabie + glycated hemoglobin | 1.00 | 0.22 (0.09-0.50) | 0.06 (0.02-0.19) | 0.12 (0.04-0.37) | <0.001 |
| Multivariable (excluding first 3 yr of | 1.00 | 0.16 (0.06-0.38) | 0.04 (0.01-0.12) | 0.06 (0.02-0.19) | <0.001 |
| follow-up) | | | | | |
| Men | | | | | |
| Median SHBG level-nmol/liter (range) | 15.2 (4.4-19.4) | 22.2 (19.4-25.7) | 29.7 (25.8-33.9) | 38.0 (34.2-75.7) | |
| No. of participants-case patients/controls | 92/43 | 47/42 | 24/43 | 7/42 | |
| Simple model 1-odds ratio (95% CI) | 1.00 | 0.62 (0.31-1.23) | 0.36 (0.16-0.82) | 0.11 (0.03-0.37) | <0.001 |
| Multivariable model 2-odds ratio (95% CI) | 1.00 | 0.48 (0.22-1.03) | 0.41 (0.15-1.14) | 0.10 (0.03-0.36) | <0.001 |
| Sensitivity model-odds ratio (95% CI) | | | | | |
| Multivariable + glycated hemoglobin | 1.00 | 0.39 (0.08-1.97) | 0.21 (0.01-3.37) | 0.10 (0.01-0.75) | 0.02 |
| Multivariable (excluding first 2 yr of follow-up) | 1.00 | 0.63 (0.26-1.53) | 0.38 (0.11-1.37) | 0.08 (0.01-0.69) | <0.001 |

*Simple model 1 was adjusted for body-mass index (BMI) and three matching factors: age, self-reported race, and fasting status. For women, multivariable model 2 was adjusted for BMI; the three matching factors; smoking status; alcohol use; degree of exercise; presence or absence of hypertension, family history of diabetes, past use of hormone-replacement therapy, and multivitamin use; years of oral contraceptive use and years since menopause; and cause of menopause. Sensitivity models for women were adjusted as for model 2 plus age at menarche, number of pregnancies, age at first pregnancy of ≧6 months' gestation, marital status, and educational level. For men, multivariable model 2 was adjusted for BMI; the three matching factors; smoking status; degree of exercise; alcohol use; presence or absence of multivitamin use, hyperlipidemia, and family history of diabetes; and blood pressure. HDL denotes high-density lipoprotein, and LDL low-density lipoprotein.
†Data for two controls were missing and were not included.

TABLE 5
Subgroup analysis of plasma SHBG and type 2 diabetes risk.

| Characteristics* | SHBG | |
|---|---|---|
| | (+1 logSD, per 60% increase) | |
| | | P for |
| Women | OR (95% CI) | interaction |
| Body Mass Index † | | |
| BMI <25 | 0.36 (0.21-0.62) | P = 0.85 |
| EMI 25-29.9 | 0.31 (0.19-0.52) | |
| BMI ≧30 | 0.29 (0.17-0.50) | |
| Past Postmenopausal Hormone Use | | |
| Never | 0.28 (0.17-0.44) | P = 0.27 |
| Past | 0.38 (0.25-0.59) | |
| Years Since Menopause | | |
| <5 years | 0.35 (0.19-0.63) | P = 0.92 |
| 5-10 years | 0.29 (0.16-0.53) | |
| >10 years | 0.31 (0.20-0.50) | |
| Hyperlipidernia | | |
| No | 0.32 (0.21-048) | P = 0.77 |
| Yes | 0.29 (0.18-0.48) | |
| Family History of Diabetes | | |
| No | 0.32 (0.22-0.47) | P = 0.84 |
| Yes | 0.30 (0.17-0.53) | |
| | (+1 logSD, per 48% increase) | |
| | | P for |
| Men | OR (95% GI) | Interaction |
| Age | | |
| <60 yrs | 0.31 (0.15-0.63) | P = 0.37 |
| ≧60 yrs | 0.47 (0.27-0.83) | |
| Body Mass Index† | | |
| BMI <25 | 0.76 (0.29-2.01) | P = 0.25 |
| BMI 25-29.9 | 0.29 (0.15-0.55) | |
| BMI ≧30 | 0.22 (0.07-0.73) | |
| Hyperlipidemia | | |
| No | 0.61 (0.33-1.10) | P = 0.05 |
| Yes | 0.26 (0.13-0.50) | |
| Family History of Diabetes | | |
| No | 0.32 (0.19-0.55) | P = 0.10 |
| Yes | 0.67 (0.32-1.41) | |

In genotype analyses, neither the rs6257 nor the rs6259 SNP had genotype distributions deviating from Hardy-Weinberg equilibrium among controls. The pairwise linkage disequilibrium between rs6257 and rs6259 was minimal (r2=0.13, P=0.02). Although the rs6257 and rs6259 variants explained 2.2% of the variance in levels of sex hormone-binding globulin, this statistic is not the only measure of instrument strength. Most importantly, carriers of an rs6257 variant allele (CC or CT) had a 10% lower level of sex hormone-binding globulin than the wild-type homozygotes (TT) (P=0.004), and carriage of a variant allele appeared to increase the risk of type 2 diabetes among both men and women. In contrast, carriers of an rs6259 variant allele (AA or AG) had a 10% higher level of sex hormone-binding globulin (P=0.005) and a lower risk of type 2 diabetes (Table 6).

TABLE 6
Plasma Sex Hormone-Binding Globulin (SHBG) levels and risk of type 2 diabetes
among the study participants, according to polymorphisms in the SHBG gene.

| | SHBG Genotype | | | | | |
|---|---|---|---|---|---|---|
| | rs6259 | | | rs6257 | | |
| | GG | AG or AA | | TT | CT or CC | |
| Variable | (wild-type) | (variant) | P value | (wild-type) | (variant) | P value |

SHBG level

| | | | | | | |
|---|---|---|---|---|---|---|
| Relative mean difference women and | 1.00 | 1.10 (1.03-1.18) | 0.005 | 1.00 | 0.90 (0.84-0.97) | 0.004 |
| men - odds ratio (95% CI) | | | | | | |

Women only

| | | | | |
|---|---|---|---|---|
| Mean (95% CI) - nmol/liter | 25.1 (24.0-26.1) | 27.1 (25.2-29.3) | 26.3 (25.2-27.3) | 23.4 (21.5-25.4) |
| Relative mean difference - odds ratio | 1.00 | 1.08 (0.99-1.18) | 1.00 | 0.89 (0.81-0.98) |
| (95% CI) | | | | |

Men only

| | | | | |
|---|---|---|---|---|
| Mean (95% CI) - nmol/liter | 21.8 (20.7-23.0) | 24.7 (22.6-27.0) | 22.8 (21.7-23.9) | 20.9 (19.1-22.8) |
| Relative mean difference - odds ratio | 1.00 | 1.13 (1.02-1.25) | 1.00 | 0.92 (0.83-1.01) |
| (95% CI) | | | | |

Risk of type 2 diabetes, women and men -
odds ratio (95% CI)†

| | | | | | | |
|---|---|---|---|---|---|---|
| Model 1 | 1.00 | 0.66 (0.47-0.93) | 0.02 | 1.00 | 1.39 (0.94-2.04) | 0.09 |
| Model 2 | 1.00 | 0.68 (0.45-1.02) | 0.06 | 1.00 | 1.68 (1.07-2.64) | 0.02 |

*The relative mean difference is the ratio of the means between the variant and wild-type genotypes. P > 0.50 for all comparisons between women and men.
†Model 1 was adjusted for body-mass index (BMI) and three matching factors: age, self-reported race, and fasting status. Model 2 was adjusted for BMI and the three matching factors, as well as smoking status; alcohol use; degree of exercise; presence or absence of history of hypertension, family history of diabetes, and multivitamin use; plus (in women) presence or absence of past hormone-replacement therapy and years of oral contraceptive use.

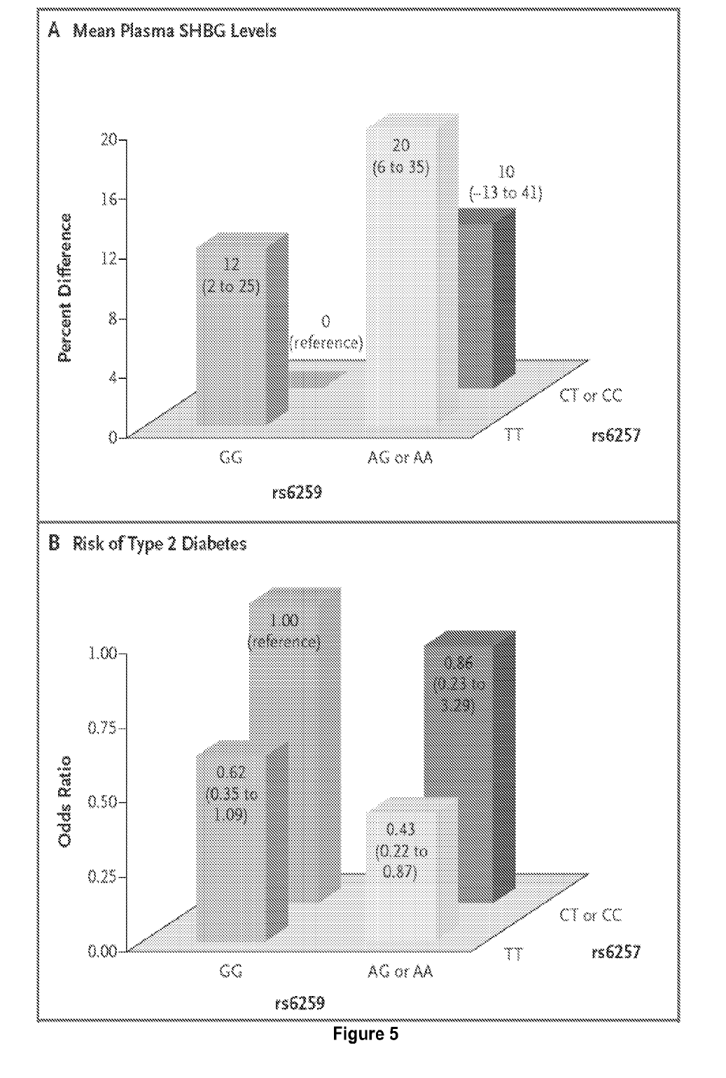

Owing to the low linkage disequilibrium between these two variant SNPs, we also conducted a joint association analysis involving stratification on the basis of the genotypes of rs6257 and rs6259, rather than a haplotype analysis. The findings indicated the independent and additive effects of rs6257 and rs6259 on levels of sex hormone-binding globulin and on the risk of type 2 diabetes (FIG. 5). The presence of variant alleles of both SNPs yielded a difference of 20% (95% CI, 6 to 35) in levels of sex hormone-binding globulin. Furthermore, consistent with differences in levels of sex hormone-binding globulin, participants with the rs6257 wild-type genotype (TT) and a rs6259 variant genotype (AG or AA) (reflecting 21.4% of controls) had a lower risk of type 2 diabetes than those carrying an rs6257 variant allele (CT or CC genotype) and the rs6259 wild-type genotype (GG) (representing 15.6% of controls) (odds ratio, 0.43; 95% CI, 0.22 to 0.87) (FIG. 5). No association was observed between SHBG polymorphisms and BMI in these two cohorts, indicating that the effects of the SHBG gene on the risk of type 2 diabetes may be independent from the effect of BMI.

We next used rs6257 and rs6259 alleles as instruments in mendelian randomization analysis. According to the mendelian law of independent assortment, genetic variants should be distributed independently and randomly with respect to other genetic variants, assuming no linkage disequilibrium or population stratification (Smith G D, et al. (2007) PLoS Med 4:e352-e352; Lawlor D A, et al. (2008) Stat Med 27:1133-1163). If environmental and lifestyle covariates are evenly distributed at baseline across SHBG genotypes, genetic variants may be used as randomization instruments to estimate potential causal associations, in this case between levels of sex hormone-binding globulin and the risk of type 2 diabetes. As instrumental variables, the rs6257 and rs6259 alleles seem to satisfy the three main criteria as genetic variants in mendelian randomization analysis: the genotypes should be robustly associated with the intermediate phenotype, should not be associated with confounding factors that may bias the association between the intermediate phenotype and disease outcome (see Table 7), and should exert their effect on the clinical outcome only through the specific intermediate phenotype (Sheehan N A, et al. (2008) PLoS Med 5:e177-e177). If all these assumptions are satisfied, the coefficient estimates based on the use of SHBG genotypes as instruments would be unconfounded (Lawlor D A, et al. (2008) Stat Med 27:1133-1163; Thomas D C, Conti D V (2004) Int J Epidemiol 33:21-25). The mendelian randomization estimate was computed from the ratio of the coefficient of the association between genotype and disease to that of the association between genotype and sex hormone-binding globulin levels; the estimate reflects the potential causal effect of sex hormone-binding globulin levels on the risk of type 2 diabetes (Smith G D, et al. (2007) PLoS Med 4:e352-e352).

TABLE 7
Characteristics by SHBG polymorphisms among controls in women*.

| | rs6257 | | | rs6259 | | |
|---|---|---|---|---|---|---|
| Characteristics (median, or %) | TT | CT and CC | P value | GG | AG and AA | P value |
| Age | 60.5 | 60.7 | 0.56 | 60.0 | 62.2 | 0.07 |
| Body Mass Index | 24.8 | 25.4 | 0.88 | 24.9 | 24.8 | 0.80 |
| Alcohol Intake (g/day) | 0.86 | 0.43 | 0.15 | 0.86 | 0.86 | 0.29 |
| Current smoking (%) | 12 | 24 | 0.06 | 15 | 14 | 0.97 |
| Physical activity (% ≧once/wk) | 41 | 34 | 0.56 | 42 | 31 | 0.26 |
| Past menopausal hormone use (%) | 29 | 23 | 0.28 | 26 | 36 | 0.08 |
| Pregnancies, ≧5 (%) | 34 | 40 | 0.74 | 34 | 37 | 0.95 |
| Cause of menopause (% natural) | 69 | 63 | 0.38 | 70 | 60 | 0.12 |
| History of hypertension (%) | 32 | 23 | 0.13 | 30 | 33 | 0.54 |
| Family history of diabetes (%) | 24 | 34 | 0.10 | 29 | 16 | 0.02^{†} |

*Among controls not missing genotyping data
^{†}Due to genetic inheritance, there is an expected automatic correlation between shared genotypes and family history; this does not bias the results, while in fact, it supports the role of this SNP in diabetes risk.

We ascertained that the predicted odds ratio of type 2 diabetes per natural-log standard-deviation increase in the level of sex hormone-binding globulin was 0.28 (95% CI, 0.13 to 0.58) in women and 0.29 (95% Cl, 0.15 to 0.58) in men (Table 8). These highly concordant estimates were virtually identical to odds ratios obtained through conventional multivariable analyses and were consistently observed in both women and men, indicating negligible residual confounding of sex hormone-binding globulin level and risk of type 2 diabetes in these two cohorts.

TABLE 8
Odds Ratios for type 2 diabetes per Unit of Increase in the
Sex Hormone-Binding Globulin (SHBG) Level*.

| Analysis | Odds Ratio (95% CI) |
|---|---|
| Mendelian randomization analysis† | |
| Allele rs6259 and rs6257 | |
| Women | 0.28 (0.13-0.58) |
| Men | 0.29 (0.15-0.58) |
| Allele rs6259 | |
| Women and men‡ | 0.23 (0.10-0.54) |
| Women only | 0.19 (0.04-1.01) |
| Men only | 0.25 (0.09-0.65) |
| Allele rs6257 | |
| Women and men‡ | 0.40 (0.19-0.88) |
| Women only | 0.39 (0.17-0.88) |
| Men only§ | 0.53 (0.05-5.22) |
| Conventional multivariable analysis¶ | |
| Women | |
| Simple model 1 | 0.40 (0.31-0.51) |
| Multivariable model | 0.34 (0.26-0.45) |
| Men | |
| Simple model 1 | 0.43 (0.31-0.59) |
| Multivariable model | 0.39 (0.27-0.58) |

*The unit of increase measured was the natural-log standard-deviation in sex-specific control distributions.
†The mendelian randomization analyses involved SHBG genotypes as instrumental variables in multivariable generalized linear models. [symbol]
‡P = 0.78 and P = 0.81 for the difference between men and women in carriage of a variant rs6259 allele and carriage of a variant rs6257 allele, respectively. All P = 0.001 for mendelian instrument rs6259 and for joint analyses. P = 0.02 for mendelian instrument rs6257, and P < 0.001 for the conventional muitivariable analyses.
§The variant of the odds ratio associated with the rs6257 variant, for men only, was calculated with the use of a robust variance estimator.
¶Conventional models were adjusted for covariates as described for each model in Table 3. [symbol]
indicates data missing or illegible when filed

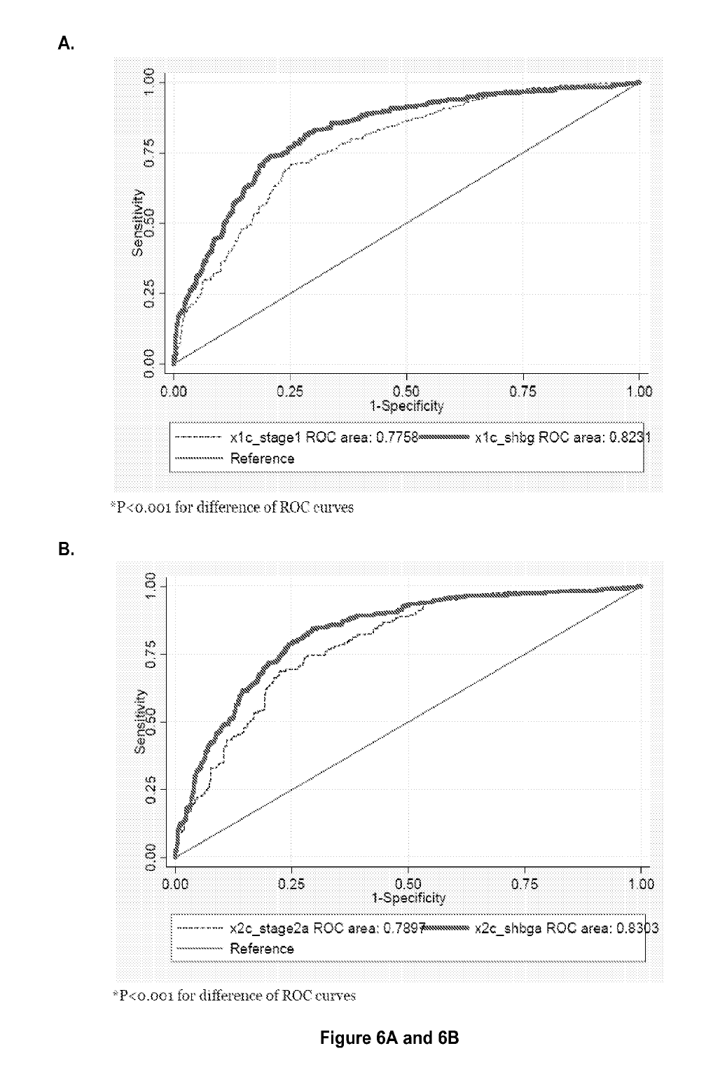

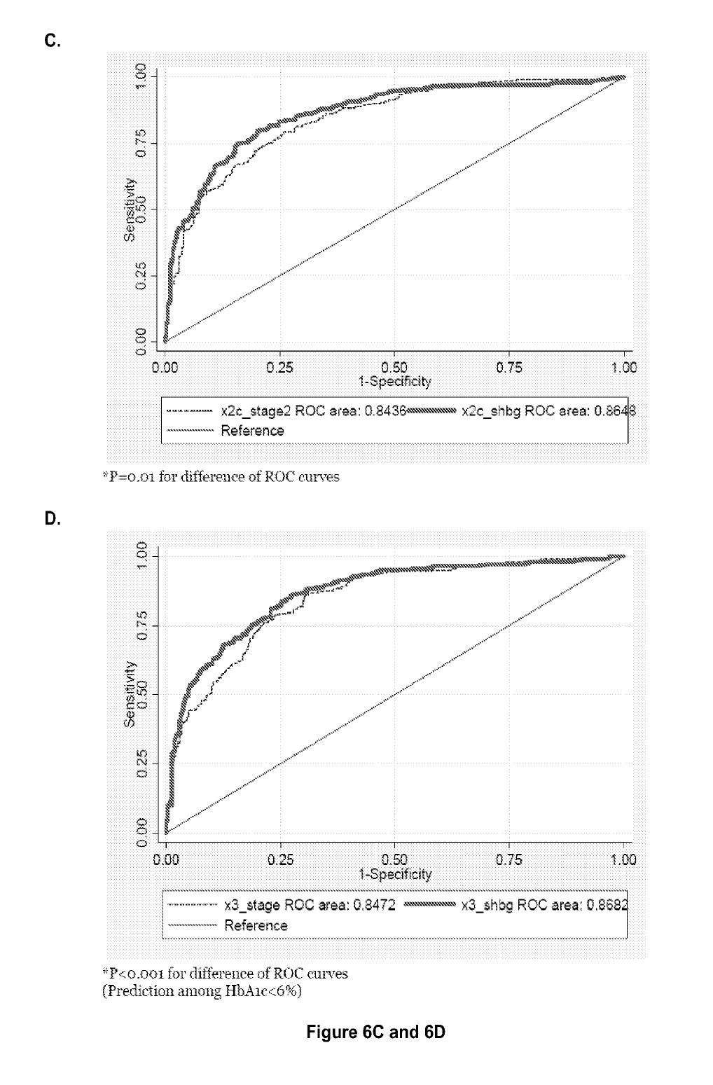

We further conducted a relative receiver-operating-characteristic analysis to determine whether the level of sex hormone-binding globulin could classify case patients with type 2 diabetes and controls more accurately than multiple established risk factors. Comparative ROC curves and C-statistics of adding sex hormone-binding globulin to various multivariable prediction models was determined. Plasma sex hormone-binding globulin improved the relative prediction of type 2 diabetes in all models (FIG. 6, p<0.001 for all comparisons), including the base model comprising traditional risk factors (FIG. 6A), an expanded model comprising traditional risk factors plus C-protein (CRP) (FIG. 6B), an expanded model comprising traditional risk factors plus glycated hemoglobin (HbA1c) (FIG. 6C), and a comprehensive model that included traditional risk factors, CRP, and glycated hemoglobin (C-statistic 0.87 vs. 0.5, p<0.001)(FIG. 6D).

### Discussion

There are four main findings regarding sex hormone-binding globulin and the risk of type 2 diabetes from these two prospective studies. First, the risk of type 2 diabetes among participants with sex hormone-binding globulin levels in the highest quartile was only one tenth the risk among those with levels in the lowest quartile. Second, the rs6257 and rs6259 SHBG polymorphisms were consistently associated with levels of sex hormone-binding globulin and were predictive of risk of type 2 diabetes in directions corresponding to their effects on plasma sex hormone-binding globulin levels. Third, levels of the globulin have a predictive ability for the risk of type 2 diabetes beyond that of traditional risk factors, including glycated hemoglobin and CRP. Finally, the strong relation between levels of sex hormone-binding globulin and risk of type 2 diabetes was confirmed both in standard multivariable analyses and in mendelian randomization analyses in which suitable genetic variants were used as randomization instruments.

These findings demonstrate a causal relationship between sex hormone-binding globulin polymorphisms, sex hormone-binding globulin levels, and type 2 diabetes. Although reverse causation has been suggested by results of cross-sectional studies in which prediabetic hyperinsulinemia is thought to inhibit the production of sex hormone-binding globulin (Haffner S M. (1996) Horm Res 45:233-237; Kalme T, et al. (2003) J Steroid Biochem Mol Biol 86:197-200), we prospectively studied participants who were apparently healthy at baseline, thus establishing a basis for detection of a temporal relationship. Potential reverse causation from undiagnosed diabetes may be a concern in our work. However, all participants were health professionals, with more valid diagnostic information and higher screening rates than those in the general population. Moreover, exclusion of the first few years of follow-up data in both studies did not affect our results, decreasing the likelihood of reverse causation. Our findings remained robust in multiple sensitivity analyses restricted to participants with glycated hemoglobin values of less than 6%. Overall, the strength and robustness of the associations indicate that residual confounding and reverse causation are unlikely.

Furthermore, the identification of SHBG germ-line variants affecting the risk of type 2 diabetes allowed us to use Mendelian randomization, rather than just conventional multivariable methods, to account for potential biases due to residual confounding and reverse causation. The estimates of genotype-disease association appear to support the plausibility of a causal relationship between levels of sex hormone-binding globulin and type 2 diabetes. Analyses of data from women and men in our study yielded consistent associations among SHBG SNPs, intermediate phenotypes, and type 2 diabetes: carriage of variant rs6257 was associated with lower levels of sex hormone-binding globulin and higher risk of type 2 diabetes, whereas carriage of rs6259 was associated with higher levels of sex hormone-binding globulin and lower risk of type 2 diabetes. Elevated levels of circulating sex hormone-binding globulin among carriers of an rs6259 variant allele warrant further functional studies; the elevation may be due to an amino acid substitution of asparagine for aspartic acid (D356N) at rs6259. This locus is an N-glycosylation consensus site that alters the binding of sex hormone-binding globulin to membrane receptors and other proteins and reduces its clearance from the circulation, resulting in higher levels of the globulin (Berndt S I, et al. (2007) Cancer Epidemiol Biomarkers Prey 16:165-168). The associations found for rs6257, a SNP that flanks, and is located 17 bp upstream of, exon 2 also suggests the presence of potential key splicing or regulatory elements in that region. Since SHBG genetic variants may exert their effects across carriers' lifetimes, the strong odds ratios relating levels of sex hormone-binding globulin to the risk of type 2 diabetes obtained from our mendelian randomization analysis may represent the average lifetime risk attributable to sex hormone-binding globulin alone, independent of traditional risk factors.

We demonstrated that two SHBG SNPs were suitable randomization instruments for elucidating the relation between levels of sex hormone-binding globulin and the risk of type 2 diabetes. Although residual confounding, particularly by adiposity, is possible with conventional observational analysis of biomarkers, levels of sex hormone-binding globulin were only modestly correlated with adiposity, and these results were robust even after dual adjustment for BMI and waist circumference. More importantly, these interrelationships among genotypes, plasma protein levels, and phenotypes of type 2 diabetes outcomes were consistently observed in two independent cohorts.

In conclusion, our prospective studies of postmenopausal women and men showed that higher levels of circulating sex hormone-binding globulin were strongly associated, indeed causally associated, with a decreased risk of type 2 diabetes. Two germ-line variants in the SHBG gene were also identified as being directly associated with both levels of sex hormone-binding globulin and the risk of type 2 diabetes. These strong and consistent findings, obtained with the use of multiple analytic approaches and subgroup analyses in two independent cohorts, demonstrate that sex hormone-binding globulin plays an important role in the development of type 2 diabetes at both the genomic and phenotypic levels and that sex hormone-binding globulin is an important target in stratification for the risk of type 2 diabetes and early intervention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of predicting a subject's susceptibility to and risk of developing type 2 diabetes, said method comprising:
obtaining a sex hormone-binding globulin (SHBG) level value for said subject; and
predicting said subject's susceptibility to and risk of developing type 2 diabetes from said obtained SHBG level value.

2. The method according to claim 1, wherein said SHBG level value is obtained by obtaining a fluid sample from said subject and determining SHBG concentration in said fluid sample.

3. The method according to claim 2, further comprising comparing the SHBG concentration to a reference value to obtain a comparison and characterizing said subject's susceptibility to and risk of developing type 2 diabetes in the future based upon said comparison.

4. The method according to claim 3, wherein said reference value is an SHBG concentration of about 24 nmol/liter of plasma in women and about 19 nmol/liter of plasma in men.

5. The method according to claim 4, wherein a subject with an SHBG concentration that is greater than the reference value has a decreased susceptibility to and risk of developing diabetes.

6. The method according to claim 5, wherein the subject is a woman and is determined to be at least 6 times less likely to develop type 2 diabetes than a woman with an SHBG concentration of 24 nmol/liter or less.

7. The method according to claim 5, wherein the subject is a man and is determined to be at least about 2 times less likely to develop type 2 diabetes than a man with an SHBG concentration of 19 nmol/liter or less.

8. The method according to claim 2, wherein said fluid sample is a blood sample.

9. The method of claim 1, wherein said individual is an apparently healthy, non-smoking individual.

10. The method according to claim 1, wherein said SHBG level value is obtained by genotyping said subject for a SHBG polymorphism selected from the group consisting of, rs6257 and rs6259.

11. The method according to claim 10, wherein said SHBG polymorphism is rs6257 and said method comprises predicting an increased susceptibility to and risk of developing type 2 diabetes if said subject carries at least one C allele of said rs6257 polymorphism.

12. The method according to claim 11, wherein said subject's predicted susceptibility to and risk of develop type 2 diabetes is about 1.4 times more likely than an individual that is wild type for rs6257.

13. The method according to claim 10, wherein said SHBG polymorphism is rs6259 and said method comprises predicting a decreased susceptibility to and risk of developing type 2 diabetes if said subject carries at least one A allele of said rs6259 polymorphism.

14. The method according to claim 13, wherein said subject's predicted susceptibility to and risk of develop type 2 diabetes is about 1.5 times less likely than an individual that is wild type for rs6259.

15. The method according to claim 1, wherein said obtaining a sex hormone-binding globulin (SHBG) level value for said subject comprises both determining a sample SHBG concentration and genotyping said subject for a SHBG polymorphism.

16. The method according to claim 1, wherein said method further comprises evaluating said subject for the presence of one or more additional type 2 diabetes prediction factors selected from the group consisting of Body Mass Index, smoking status, alcohol use, degree of exercise, history of hypertension, family history of diabetes, multivitamin use, past hormone-replacement therapy (in women), years of oral contraceptive use (in women), years since menopause (in women), cause of menopause (in women), waist circumference, amount of C-reactive protein (CRP), amount of glycated hemoglobin (e.g., HbA1c), testosterone levels, and estradiol levels.

17. A method for predicting the likelihood that a subject will benefit from a treatment for reducing the risk of type 2 diabetes, the method comprising obtaining a SHBG level value for said subject.

18. The method according to claim 17, wherein said SHBG level value is obtained by obtaining a fluid sample from said subject and determining SHBG concentration in said fluid sample.

19. The method according to claim 18, further comprising comparing SHBG concentration to a reference value to obtain a comparison and characterizing said subjects' susceptibility to and risk of developing type 2 diabetes in the future based upon said comparison.

20. The method according to claim 19, wherein said reference value is about 24 nmol/liter of plasma in women and 19 nmol/liter of plasma in men.

21. The method according to claim 20, wherein a subject with an SHBG concentration that is greater than the reference value is more likely to benefit from a treatment for reducing the risk of type 2 diabetes.

22. The method according to claim 17, wherein said SHBG level value is obtained by genotyping said subject for an SHBG polymorphism that is rs6257.

23. The method according to claim 22, wherein the method comprises determining that a subject is more likely to benefit from a treatment for reducing the risk of type 2 diabetes when the subject carries at least one C allele of said rs6257 polymorphism.

24. The method according to claim 17, wherein said SHBG level value is obtained by genotyping said subject for an SHBG polymorphism that is rs6259.

25. The method according to claim 24, wherein the method comprises determining that a subject is more likely to benefit from a treatment for reducing the risk of type 2 diabetes when the subject carries two G alleles of said rs6259 polymorphism.

26. A device configured to obtain a sex hormone-binding globulin (SHBG) level value for a sample from a subject and output a prediction of said subject's susceptibility to and risk of developing type 2 diabetes based on said obtained sex hormone-binding globulin (SHBG) level value.

27. The device according to claim 26, wherein said device comprises a ligand that specifically binds to SHBG.

28. The device according to claim 27, wherein said ligand is an antibody or binding fragment thereof.

29. The device according to claim 26, wherein said device is configured to genotype said subject for a SHBG polymorphism.

30. The device according to claim 29, wherein said SHBG polymorphism is rs6257.

31. The device according to claim 29, wherein said SHBG polymorphism is rs6259.

32. A device configured to obtain a sex hormone-binding globulin (SHBG) level value for a sample from a subject, wherein said device comprises:
a ligand that specifically binds to SHBG; and
an SHBG polymorphism detection element.

33. The device according to claim 32, wherein said ligand is an antibody or binding fragment thereof.

34. A method of inhibiting the development of type 2 diabetes in a subject, said method comprising administering to said subject an agent that increases at least one of SHBG concentration levels and SHBG activity levels in said subject in an amount effective to inhibit the development of type 2 diabetes in said subject.

35. The method according to claim 34, wherein said agent is a nucleic acid agent.

36. The method according to claim 34, wherein said agent is a peptide agent.

37. The method according to claim 34, wherein said agent is a small molecule.

38. A method of treating a subject for type 2 diabetes, said method comprising administering to said subject an agent that increases at least one of SHBG concentration levels and SHBG activity levels in said subject in an amount effective to treat said subject for type 2 diabetes.
